(19) United States
(12) Patent Application Publication
(10) Pub. No.: US 2013/0039974 A1
(43) Pub. Date: Feb. 14, 2013

(54) ANTI-MUC1 ANTIBODIES FOR CANCER DIAGNOSTICS

(71) Applicants: Donald W. Kufe, Wellesley, MA (US); Surender Kharbanda, Natick, MA (US)

(72) Inventors: Donald W. Kufe, Wellesley, MA (US); Surender Kharbanda, Natick, MA (US)

(21) Appl. No.: 13/572,538

(22) Filed: Aug. 10, 2012

Related U.S. Application Data
(60) Provisional application No. 61/522,144, filed on Aug. 10, 2011.
(60) Provisional application No. 61/537,391, filed on Sep. 21, 2011.

(51) Int. Cl. A61K 39/395; C07K 16/30; A61P 35/02; A61K 39/44; A61K 9/127; G01N 33/574; A61P 35/00; B82Y 5/00

(52) U.S. Cl. 424/450; 435/7.23; 530/388.8; 424/155.1; 424/135.1; 424/133.1; 424/178.1; 977/774; 977/906; 977/907; 977/797

(57) ABSTRACT

The present invention is directed to antibodies binding to the MUC1 cytoplasmic domain and methods of using such antibodies to treat, diagnose, detect and monitor cancers that express the MUC1 antigen.

The present application claims benefit of U.S. Provisional Application Ser. Nos. 61/537,391, filed Sep. 21, 2011, and 61/522,114, filed Aug. 10, 2011, the entire contents of both applications hereby being incorporated by reference.

This invention was made with government support under grant number CA 97098 awarded by The National Cancer Institute. The government has certain rights in the invention.

### BACKGROUND OF THE INVENTION

1. Field of the Invention

The present invention relates generally to the fields of medicine, oncology and immunodiagnostics. More particularly, it concerns the development of immunoreagents for use in diagnosing detecting and monitoring MUC1-positive cancers.

2. Background of the Invention

Mucins are extensively O-glycosylated proteins that are predominantly expressed by epithelial cells. The secreted and membrane-bound mucins form a physical barrier that protects the apical borders of epithelial cells from damage induced by toxins, microorganisms and other forms of stress that occur at the interface with the external environment. The transmembrane mucin 1 (MUC1) can also signal to the interior of the cell through its cytoplasmic domain. MUC1 has no sequence similarity with other membrane-bound mucins, except for the presence of a sea urchin sperm protein-enterokinase-agrin (SEA) domain (Duraisamy et al., 2006). In that regard, MUC1 is translated as a single polypeptide and then undergoes autocleavage at the SEA domain (Macao, 2006).

MUC1 has been studied extensively by the inventors and others for its role in cancer. As discussed above, human MUC1 is heterodimeric glycoprotein, translated as a single polypeptide and cleaved into N- and C-terminal subunits in the endoplasmic reticulum (Ligtenberg et al., 1992; Macao et al., 2006; Levitin et al., 2005). Aberrant overexpression of MUC1, as found in most human carcinomas (Kufe et al., 1984), confers anchorage-independent growth and tumorigenicity (Li et al., 2003a; Huang et al., 2003; Schroeder et al., 2004; Huang et al., 2005). Other studies have demonstrated that overexpression of MUC1 confers resistance to apoptosis induced by oxidative stress and genotoxic anti-cancer agents (Yin and Kufe, 2003; Ren et al., 2004; Raina et al., 2004; Yin et al., 2004; Raina et al., 2006; Yin et al., 2007).

The family of tethered and secreted mucins functions in providing a protective barrier of the epithelial cell surface. With damage to the epithelial layer, the tight junctions between neighboring cells are disrupted, and polarity is lost as the cells initiate a heregulin-induced repair program (Vermeer et al., 2003). MUC1-N is shed from the cell surface (Abe and Kufe, 1989), leaving MUC1-C to function as a transducer of environmental stress signals to the interior of the cell. In this regard, MUC1-C forms cell surface complexes with members of the ErbB receptor family, and MUC1-C is targeted to the nucleus in the response to heregulin stimulation (Li et al., 2001; Li et al., 2003c). MUC1-C also functions in integrating the ErbB receptor and Wnt signaling pathways through direct interactions between the MUC1 cytoplasmic domain (CD) and members of the catenin family (Huang et al., 2005; Li et al., 2003c; Yamamoto et al., 1997; Li et al., 1998; Li et al., 2001; Li and Kufe, 2001). Other studies have demonstrated that MUC1-CD is phosphorylated by glycogen synthase kinase 3β, c-Src, protein kinase Cδ, and c-Abl (Raina et al., 2006; Li et al., 1998; Li et al., 2001; Ren et al., 2002).

The mechanisms responsible for nuclear targeting of MUC1-C are unclear. Proteins containing a classical nuclear localization signal (NLS) are imported into the nucleus by first binding to importin α and then, in turn, importin β (Weis, 2003). The cargo-importin α/β complex docks to the nuclear pore by binding to nucleoporins and is transported through the pore by a mechanism dependent on the Ran GTPase. Classical NLSs are monopartite with a single cluster of 4-5 basic amino acids or bipartite with two clusters of basic amino acids separated by a linker of 10-12 amino acids. MUC1-CD contains a RRK motif that does not conform to a prototypical monopartite NLS (Hodel et al., 2002). However, certain proteins containing non-classical NLSs are transported through the nuclear pore by binding directly to importin β (Kau et al., 2004). Importin β associates with several nucleoporins (Ryan and Wente, 2000), including Nup62, which is located on both the cytoplasmic and nucleoplasmic faces of nuclear pore complexes (Percipalle et al., 1997). Other studies have indicated that β-catenin is imported into the nucleus by an importin- and nucleoporin-independent mechanism (Suh and Gumbiner, 2003).

In 2006, the inventors reported that MUC1 is imported into the nucleus by a mechanism involving binding to Nup62 (Leng et al., 2007). They also demonstrate that MUC1 forms oligomers through a CQC motif in the MUC1 cytoplasmic domain and that MUC1 oligomerization is necessary for nuclear import. In 2007, they also demonstrated that overexpression of MUC1 in human carcinoma cells is associated with constitutive activation of NF-κB p65 (Ahmad et al. 2007). MUC1 was shown to interact with the high-molecular-weight IκB kinase (IKK) complex in vivo, and that the MUC1 cytoplasmic domain binds directly to IKKβ and IKKγ. Interaction of MUC1 with both IKKβ and IKKγ is necessary for IKKβ activation, resulting in phosphorylation and degradation of IKBα. These findings indicated that MUC1 is important for physiological activation of IKKβ and that overexpression of MUC1, as found in human cancers, confers sustained induction of the IKKβ-NF-κB p65 pathway.

### SUMMARY OF THE INVENTION

Thus, in accordance with the present invention, there is provided a method of detecting MUC1 in a cell or tissue comprising (a) contacting the cell or tissue with an antibody reagent that binds immunologically to the cytoplasmic domain of MUC1; and (b) detecting the antibody reagent bound to the cell or tissue. The cell or tissue may be a cancer cell or tissue, such as a tumor biopsy, and may also be a MUC1-positive cell, tissue or biopsy. The cell, tissue or tumor biopsy may be from a patient not having been previously diagnosed with cancer, or from a patient been previously diagnosed with cancer, be that a MUC1-positive cancer or MUC1-negative cancer. The tumor biopsy may also be from a patient undergoing or having previously undergone cancer treatment. In any of the foregoing embodiments, the methods may further comprise contacting a second cell or tissue from a second tumor biopsy with the reagent. The method may further comprise making a treatment decision for the subject based on the results of the detection or further comprising providing a cancer treatment to the subject.

The reagent may be an antibody or antibody fragment, such as recombinant antibody, including a single chain antibody, a single domain antibody. The cancer cell or tissue may be a solid tumor cell or tissue, such as lung cancer, brain cancer, head & neck cancer, breast cancer, skin cancer, liver cancer, pancreatic cancer, stomach cancer, colon cancer, rectal cancer, uterine cancer, cervical cancer, ovarian cancer, testicular cancer, skin cancer, or esophageal cancer. The cancer cell may be a leukemia or myeloma, such as acute myeloid leukemia, chronic myelogenous leukemia or multiple myeloma. The reagent may be labeled with a detectable moiety, such as a chemilluminscent, a chromophore, a fluorophore, a magnetic particle, a dye, a radiolabel, an enzyme or a ligand/ligand binding agent. The may not be not labeled, and detecting is accomplished using a secondary binding agent that is labeled with a detectable moeity, the secondary binding agent having affinity for the antibody reagent. Detection may comprise a ELISA or RIA, a competitive assay, or immunohistochemistry.

The reagent may be an antibody or antibody fragment comprising (a) a light chain variable sequence according to SEQ ID NO:7 or a sequence having 80% identity to SEQ ID NO:7, and a heavy chain sequence according to SEQ ID NO:2 or a sequence having 80% identity to SEQ ID NO:2; or (b) a light chain variable sequence according to SEQ ID NO:16 or a sequence having 80% identity to SEQ ID NO:16, and a heavy chain sequence according to SEQ ID NO:14 or a sequence having 80% identity to SEQ ID NO:14. The antibody or antibody fragment may be encoded by (a) a light chain variable sequence according to SEQ ID NO:6 or a sequence having 70% identity to SEQ ID NO:6, and a heavy variable chain sequence according to SEQ ID NO:1 or a sequence having 70% identity to SEQ ID NO:1; or (b) a light chain variable sequence according to SEQ ID NO:15 or a sequence having 70% identity to SEQ ID NO:15, and a heavy variable chain sequence according to SEQ ID NO:13 or a sequence having 70% identity to SEQ ID NO:13. The reagent may be an antibody or antibody fragment comprising light chain variable CDR sequences according to SEQ ID NO:8-10 or 20-21, and/or an antibody or antibody fragment comprising heavy chain variable CDRs sequences according to SEQ ID NO:3-5 or 17-19.

Also provided is a monoclonal antibody comprising (a) a light chain variable sequence according to SEQ ID NO:7 or a sequence having 80% identity to SEQ ID NO:7, and a heavy chain sequence according to SEQ ID NO:2 or a sequence having 80% identity to SEQ ID NO:2; or (b) a light chain variable sequence according to SEQ ID NO:16 or a sequence having 80% identity to SEQ ID NO:16, and a heavy chain sequence according to SEQ ID NO:14 or a sequence having 80% identity to SEQ ID NO:14. The antibody or antibody fragment may be encoded by (a) a light chain variable sequence according to SEQ ID NO:6 or a sequence having 70% identity to SEQ ID NO:6, and a heavy variable chain sequence according to SEQ ID NO:1 or a sequence having 70% identity to SEQ ID NO:1; or (b) a light chain variable sequence according to SEQ ID NO:15 or a sequence having 70% identity to SEQ ID NO:15, and a heavy variable chain sequence according to SEQ ID NO:13 or a sequence having 70% identity to SEQ ID NO:13. The monoclonal antibody may comprise light chain variable CDR sequences according to SEQ ID NO:8-10 or 20-21, and/or heavy chain variable CDRs sequences according to SEQ ID NO:3-5 or 17-19. The antibodies may also be defined as comprising the CDRs (or CDR-encoding regions) of any of the foregoing sequences, which can be identified by excluding the common framework regions from the sequences.

In another embodiment, there is provided a method of treating cancer comprising contacting a MUC1-positive cancer cell in a subject with that binds immunologically to the cytoplasmic domain of MUC1. The MUC1-positive cancer cell may be a solid tumor cell, such as a lung cancer cell, brain cancer cell, head & neck cancer cell, breast cancer cell, skin cancer cell, liver cancer cell, pancreatic cancer cell, stomach cancer cell, colon cancer cell, rectal cancer cell, uterine cancer cell, cervical cancer cell, ovarian cancer cell, testicular cancer cell, skin cancer cell, or esophageal cancer cell. The MUC1-positive cancer cell may be a leukemia or myeloma, including acute myeloid leukemia, chronic myelogenous leukemia or multiple myeloma. The MUC1-positive cancer cell may be a metastatic cancer cell, a multiply drug resistant cancer cell or a recurrent cancer cell.

The method may further comprising contacting the MUC1-positive cancer cell with a second anti-cancer agent or treatment. The second anti-cancer agent or treatment may be selected from chemotherapy, radiotherapy, immunotherapy, hormonal therapy, or toxin therapy. The second anti-cancer agent may inhibit an intracellular MUC1 function. The second anti-cancer agent or treatment may be given at the same time as the first agent, or before and/or after the first agent.

The antibody may be a single chain antibody, a single domain antibody, a chimeric antibody, a Fab fragment, a recombinant antibody having specificity for the MUC1 ECD and a distinct cancer cell surface antigen, a murine antibody, including an IgG antibody, or a humanized antibody, including an IgG antibody. The antibody may further comprise an antitumor drug linked thereto, such as one linked to the antibody through a photolabile linker or an enzymatically-cleaved linker. The antitumor drug may a toxin, a radioisotope, a cytokine, or an enzyme. The antibody may further comprises a label, such as a peptide tag, an enzyme, a magnetic particle, a chromophore, a fluorescent molecule, a chemilluminescent molecule, or a dye. The antibody may be conjugated to a liposome or a nanoparticle.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Graphical representation of the data presented in Table 2.

FIG. 2. Western Blot analysis of antiserum. Antisera from both mouse 1 and mouse 2 were diluted as shown above and tested in Western Blot against GST-MUC1 (CD) as well as GST-irrelevant protein.

FIG. 3. Western blot for screening primary clones. Culture supernatants from 120 samples (15×8) we tested against 20 μg total GST MUC1 (CD) protein on the blot. Positive controls: D=DG122 (anti-GST Ab, 1:500); S=Immune Serum #2, 1:1000. Negative control: HT medium. Expected protein size: 35-38 kDa. Twenty-one positive sample were identified.

FIG. 4. Re-screening of the selected clones at 24-well stage by Western blot. Culture supernatants from selected 21 clones or were screened for reactivity against 20 μg total GST MUC1 (CD) protein on the blot or GST-irrelevant protein. Positive controls: D=DG122 (anti-GST Ab, 1:500); S1=Immune Serum #2, 1:1000; S2=Immune serum #2 for irrelevant protein. Expected protein size: 35-38 kDa. Eleven positive clones were identified.

FIG. 5. Results of subclone analysis by Western Blot. Twenty μg total GST MUC1 (CD) on the blot was used against subclosed from the eleven positive clonse. Positive controls: Anti-His Ab, (1:500); S=Immune Serum #2, 1:1000. Negative control: D-10 medium. Expected protein size: ˜13 kDa. Positive clones from 3E7: 1/20, cloning efficiency (CE)=5%; 4E5: 12/20 CE=60%; 7F6: showed smear from all clones; 8D9: 1/20 CE=5%.

FIG. 6. Analysis of MUC1-CD antibody supernatants by Western blot.

FIG. 7. Summary of the results of epitope mapping for anti-MUC1-CD antibody.

FIG. 8. Binding of His-CD to immobilized Anti-MUC1-CD (CD1, batch 1) antibody.

FIG. 9. Binding of Anti-MUC1-CD (CD1, batch 1) antibody to immobilized His-CD.

FIG. 10. Binding of Anti-MUC1-CD (CD1, batch 5) antibody to immobilized His-CD.

FIG. 11. Binding kinetics of MUC1-His-CD to immobilized anti-MUC1-CD (CD1, batch 1).

FIG. 12. Binding Kinetics of anti-MUC1-CD antibody (CD1, batch 1) to immobilized His-CD.

FIG. 13. Binding Kinetics of anti-MUC1-CD antibody (CD1, batch 5) to immobilized His-CD.

FIGS. 14-16. Immunohistochemistry of fixed human breast carcinoma tumor tissues.

### DESCRIPTION OF ILLUSTRATIVE EMBODIMENTS

The inventors have raised antibodies against the cytoplasmic domain of the MUC1 protein. The are valuable tools in general for the study of MUC1 biology in the area of cancer research and, more specifically, for use in biomarker studies as a prognostic tool for analyzing patient samples in upcoming clinical studies on anti-MUC1 therapies. This application provides information regarding the development and testing of hybridoma clones producing antibodies that are specific for MUC1 protein. These and other aspects of the invention are described in greater detail below.

### I. MUC1

A. Structure

MUC1 is a mucin-type glycoprotein that is expressed on the apical borders of normal secretory epithelial cells (Kufe et al., 1984). MUC1 forms a heterodimer following synthesis as a single polypeptide and cleavage of the precursor into two subunits in the endoplasmic reticulum (Ligtenberg et al., 1992). The cleavage may be mediated by an autocatalytic process (Levitan et al., 2005). The >250 kDa MUC1 N-terminal (MUC1 N-ter, MUC1-N) subunit contains variable numbers of 20 amino acid tandem repeats that are imperfect with highly conserved variations and are modified by O-linked glycans (Gendler et al., 1988; Siddiqui et al., 1988). MUC1-N is tethered to the cell surface by dimerization with the ˜23 kDa C-terminal subunit (MUC1 C-ter, MUC1-C), which includes a 58 amino acid extracellular region, a 28 amino acid transmembrane domain and a 72-amino acid cytoplasmic domain (CD) (Merlo et al., 1989). The human MUC1 sequence is shown below:

(SEQ ID NO: 11)
GSVVVQLTLAFREGTINVHDVETQFNQYKTEAASRYNLTISDVSVSDVPFPFSAQSGAG
VPGWGIALLVLVCVLVALAIVYLIALAVCQCRRKNYGQLDIFPARDTYHPMSEYPTY
HTHGRYVPPSSTDRSPYEKVSAGNGGSSLSYTNPAVAATSANL

The bold sequence indicates the CD, and the underlined portion is an oligomer-inhibiting peptide. With transformation of normal epithelia to carcinomas, MUC1 is aberrantly overexpressed in the cytosol and over the entire cell membrane (Kufe et al., 1984; Perey et al., 1992). Cell membrane-associated MUC1 is targeted to endosomes by clathrin-mediated endocytosis (Kinlough et al., 2004). In addition, MUC1-C, but not MUC1-N, is targeted to the nucleus (Baldus et al., 2004; Huang et al., 2003; Li et al., 2003a; Li et al., 2003b; Li et al., 2003c; Wei et al., 2005; Wen et al., 2003) and mitochondria (Ren et al., 2004).

B. Function

MUC1 interacts with members of the ErbB receptor family (Li et al., 2001b; Li et al., 2003c; Schroeder et al., 2001) and with the Wnt effector, β-catenin (Yamamoto et al., 1997). The epidermal growth factor receptor and c-Src phosphorylate the MUC1 cytoplasmic domain (MUC1-CD) on Y-46 and thereby increase binding of MUC1 and β-catenin (Li et al., 2001a; Li et al., 2001b). Binding of MUC1 and β-catenin is also regulated by glycogen synthase kinase 313 and protein kinase Cδ (Li et al., 1998; Ren et al., 2002). MUC1 colocalizes with β-catenin in the nucleus (Baldus et al., 2004; Li et al., 2003a; Li et al., 2003c; Wen et al., 2003) and coactivates transcription of Wnt target genes (Huang et al., 2003). Other studies have shown that MUC1 also binds directly to p53 and regulates transcription of p53 target genes (Wei et al., 2005). Notably, overexpression of MUC1 is sufficient to induce anchorage-independent growth and tumorigenicity (Huang et al., 2003; Li et al., 2003b; Ren et al., 2002; Schroeder et al., 2004).

Most mitochondrial proteins are encoded in the nucleus and are imported into mitochondria by translocation complexes in the outer and inner mitochondrial membranes. Certain mitochondrial proteins contain N-terminal mitochondrial targeting sequences and interact with Tom20 in the outer mitochondrial membrane (Truscott et al., 2003). Other mitochondrial proteins contain internal targeting sequences and interact with the Tom70 receptor (Truscott et al., 2003). Recent work showed that mitochondrial proteins without internal targeting sequences are delivered to Tom70 by a complex of HSP70 and HSP90 (Young et al., 2003).

### II. Producing Monoclonal Antibodies

A. General Methods

It will be understood that monoclonal antibodies binding to MUC1 will have utilities in several applications. These include the production of diagnostic kits for use in detecting and diagnosing cancer. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens or lymph nodes, or from circulating blood. The antibody-producing B lymphocytes from the immunized animal are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions. One particular murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line. More recently, additional fusion partner lines for use with human B cells have been described, including KR12 (ATCC CRL-8658; K6H6/B5 (ATCC CRL-1823 SHM-D33 (ATCC CRL-1668) and HMMA2.5 (Posner et al., 1987). The antibodies in this invention were generated using the SP2/0/mL-6 cell line, an IL-6 secreting derivative of the SP2/0 line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. Ouabain is added if the B cell source is an Epstein Barr virus (EBV) transformed human B cell line, in order to eliminate EBV transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain is also used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like.

The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the invention can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate monoclonals. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present invention include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

B. Antibodies of the Present Invention

Antibodies according to the present invention may be defined, in the first instance, by their binding specificity, which in this case is for MUC1. Those of skill in the art, by assessing the binding specificity/affinity of a given antibody using techniques well known to those of skill in the art, can determine whether such antibodies fall within the scope of the instant claims.

In one aspect, there is provided a monoclonal antibody that binds to MUC1 cytoplasmic domain. A particular type of antibody that is one that binds to an epitope defined by YGQLDIFP (SEQ ID NO: 12). Such antibodies may be produced by the clones discussed below in the Examples section using methods described herein.

In a second aspect, the antibodies may be defined by their variable sequence, which determines their binding specificity. Furthermore, the antibodies sequences may vary from the sequences provided above, optionally using methods discussed in greater detail below. For example, nucleic acid sequences may vary from those set out above in that (a) the variable regions may be segregated away from the constant domains of the light chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below). Each of the foregoing apply to the nucleic acid sequences set forth as SEQ ID NOS: 1, 6, 13 and 15, and the amino acid sequences of SEQ ID NOS: 2, 7, 14 and 16. The antibodies may also be defined by the CDRs.

C. Engineering of Antibody Sequences

In various embodiments, one may choose to engineer sequences of the identified antibodies for a variety of reasons, such as improved expression, improved cross-reactivity, diminished off-target binding or abrogation of one or more natural effector functions, such as activation of complement or recruitment of immune cells (e.g., T cells). The following is a general discussion of relevant techniques for antibody engineering.

Hybridomas may cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full length IgG antibodies were generated by subcloning heavy and light chain Fv DNAs from the cloning vector into a Lonza pConIgG1 or pConK2 plasmid vector, transfected into 293 Freestyle cells or Lonza CHO cells, and antibodies were collected an purified from the CHO cell supernatant.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

pCon Vectors™ are an easy way to re-express whole antibodies. The constant region vectors are a set of vectors offering a range of immunoglobulin constant region vectors cloned into the pEE vectors. These vectors offer easy construction of full length antibodies with human constant regions and the convenience of the GS System™

Antibody molecules will comprise fragments (such as F(ab′), F(ab′)₂) that are produced, for example, by the proteolytic cleavage of the mAbs, or single-chain immunoglobulins producible, for example, via recombinant means. Such antibody derivatives are monovalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

It may be desirable to “humanize” antibodies produced in non-human hosts in order to attenuate any immune reaction when used in human therapy. Such humanized antibodies may be studied in an in vitro or an in vivo context. Humanized antibodies may be produced, for example by replacing an immunogenic portion of an antibody with a corresponding, but non-immunogenic portion (i.e., chimeric antibodies). PCT Application PCT/US86/02269; EP Application 184,187; EP Application 171,496; EP Application 173,494; PCT Application WO 86/01533; EP Application 125,023; Sun et al. (1987); Wood et al. (1985); and Shaw et al. (1988); all of which references are incorporated herein by reference. General reviews of “humanized” chimeric antibodies are provided by Morrison (1985); also incorporated herein by reference. “Humanized” antibodies can alternatively be produced by CDR or CEA substitution. Jones et al. (1986); Verhoeyen et al. (1988); Beidler et al. (1988); all of which are incorporated herein by reference.

In related embodiments, the antibody is a derivative of the disclosed antibodies, e.g., an antibody comprising the CDR sequences identical to those in the disclosed antibodies (e.g., a chimeric, humanized or CDR-grafted antibody). In yet a further embodiment, the antibody is a fully human recombinant antibody.

Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present invention also contemplates isotype modification. By modifying the Fc region to have a different isotype, different functionalities can be achieved. For example, changing to IgG₄ can reduce immune effector functions associated with other isotypes.

Modified antibodies may be made by any technique known to those of skill in the art, including expression through standard molecular biological techniques, or the chemical synthesis of polypeptides. Methods for recombinant expression are addressed elsewhere in this document.

D. Single Chain Antibodies

A Single Chain Variable Fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alaine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present invention may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

E. Intrabody

In a particular embodiment, the antibody is a recombinant antibody that is suitable for action inside of a cell—such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. With respect to the stability, the approach is generally to either screen by brute force, including methods that involve phage diplay and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.; Persic et al., 1997).

By virtue of their ability to enter cells, intrabodies have additional uses that other types of antibodies may not achieve. In the case of the present antibodies, the ability to interact with the MUC1 cytoplasmic domain in a living cell may interfere with functions associated with the MUC1 CD, such as signaling functions (binding to other molecules) or oligomer formation. In particular, it is contemplated that such antibodies can be used to inhibit MUC1 dimer formation.

While the target for these antibodies is intracellular by nature, recent reports suggest that the large size of even fully intact antibodies is not necessarily an impediment to their efficacy. Guo et al. (2011) report that overexpressed internal tumor antigens, including those that are artificial and non-transforming (eGFP), can be targeted by intact antibodies which in turn can exhibit anti-tumor activity (inhibition of metastasis, inhibition of tumor progession, increased patient survival, reduced tumor load). Thus, while the use of intrabodies or antibody conjugates may prove useful, the modifications do not appear to be required for treatment efficacy.

F. Purification

In certain embodiments, the antibodies of the present invention may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present invention, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens my be used to simultaneously purify and select appropriate antibodies. Such methods often utilize the selection agent bound to a support, such as a column, filter or bead. The antibodies is bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. Pharmaceutical Formulations and Treatment of Cancer

A. Cancers

Cancer results from the outgrowth of a clonal population of cells from tissue. The development of cancer, referred to as carcinogenesis, can be modeled and characterized in a number of ways. An association between the development of cancer and inflammation has long-been appreciated. The inflammatory response is involved in the host defense against microbial infection, and also drives tissue repair and regeneration. Considerable evidence points to a connection between inflammation and a risk of developing cancer, i.e., chronic inflammation can lead to dysplasia.

Cancer cells to which the methods of the present invention can be applied include generally any cell that expresses MUC1, and more particularly, that overexpresses MUC1. An appropriate cancer cell can be a breast cancer, lung cancer, colon cancer, pancreatic cancer, renal cancer, stomach cancer, liver cancer, bone cancer, hematological cancer (e.g., leukemia or lymphoma), neural tissue cancer, melanoma, ovarian cancer, testicular cancer, prostate cancer, cervical cancer, vaginal cancer, or bladder cancer cell. In addition, the methods of the invention can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, and mice. Cancers may also be recurrent, metastatic and/or multi-drug resistant, and the methods of the present invention may be particularly applied to such cancers so as to render them resectable, to prolong or re-induce remission, to prevent or limit metastasis, and/or to treat multi-drug resistant cancers.

B. Formulation and Administration

The present invention provides pharmaceutical compositions comprising anti-MUC1 antibodies. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Other suitable pharmaceutical excipients include starch, glucose, lactose, sucrose, saline, dextrose, gelatin, malt, rice, flour, chalk, silica gel, sodium stearate, glycerol monostearate, talc, sodium chloride, dried skim milk, glycerol, propylene glycol, water, ethanol and the like.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

B. Cancer Therapies

As an adjunct to the diagnostic aspects of the present invention, it may be desirable to make a treatment decision based on the outcome of the diagnostic method, or to effect such a treatment. Treatment options are well known to those of skill in the art. The goal may be to kill cells, inhibit cell growth, inhibit metastasis, inhibit angiogenesis or otherwise reverse or reduce the malignant phenotype of tumor cells. Agents or factors suitable for cancer therapy include any chemical compound or treatment method that induces DNA damage when applied to a cell. Such agents and factors include radiation and waves that induce DNA damage such as, irradiation, microwaves, electronic emissions, and the like. A variety of chemical compounds, also described as “chemotherapeutic” or “genotoxic agents,” may be used. This may be achieved by irradiating the localized tumor site; alternatively, the tumor cells may be contacted with the agent by administering to the subject a therapeutically effective amount of a pharmaceutical composition.

Various classes of chemotherapeutic agents are comtemplated for use with the present invention. For example, selective estrogen receptor antagonists (“SERMs”), such as Tamoxifen, 4-hydroxy Tamoxifen (Afimoxfene), Falsodex, Raloxifene, Bazedoxifene, Clomifene, Femarelle, Lasofoxifene, Ormeloxifene, and Toremifene.

Chemotherapeutic agents contemplated to be of use, include, e.g., camptothecin, actinomycin-D, mitomycin C. The invention also encompasses the use of a combination of one or more DNA damaging agents, whether radiation-based or actual compounds, such as the use of X-rays with cisplatin or the use of cisplatin with etoposide. The agent may be prepared and used as a combined therapeutic composition, or kit, by combining it with a MUC1 peptide, as described above.

Heat shock protein 90 is a regulatory protein found in many eukaryotic cells. HSP90 inhibitors have been shown to be useful in the treatment of cancer. Such inhibitors include Geldanamycin, 17-(Allylamino)-17-demethoxygeldanamycin, PU-H71 and Rifabutin.

Agents that directly cross-link DNA or form adducts are also envisaged. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for doxorubicin, to 35-50 mg/m² for etoposide intravenously or double the intravenous dose orally. Microtubule inhibitors, such as taxanes, also are contemplated. These molecules are diterpenes produced by the plants of the genus Taxus, and include paclitaxel and docetaxel.

Epidermal growth factor receptor inhibitors, such as Iressa, mTOR, the mammalian target of rapamycin, also known as FK506-binding protein 12-rapamycin associated protein 1 (FRAP1) is a serine/threonine protein kinase that regulates cell growth, cell proliferation, cell motility, cell survival, protein synthesis, and transcription. Rapamycin and analogs thereof (“rapalogs”) are therefore contemplated for use in cancer therapy in accordance with the present invention.

Another possible therapy is TNF-α (tumor necrosis factor-alpha), a cytokine involved in systemic inflammation and a member of a group of cytokines that stimulate the acute phase reaction. The primary role of TNF is in the regulation of immune cells. TNF is also able to induce apoptotic cell death, to induce inflammation, and to inhibit tumorigenesis and viral replication.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

Other factors that cause DNA damage and have been used extensively include what are commonly known as γ-rays, x-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

In addition, it also is contemplated that immunotherapy, hormone therapy, toxin therapy and surgery can be used. In particular, one may employ targeted therapies such as Avastin, Erbitux, Gleevec, Herceptin and Rituxan.

It also should be pointed out that any of the foregoing therapies may prove useful by themselves in treating cancer.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### IV. Antibody Reagents

A. Conjugates

Antibodies may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., immunosuppression/anti-inflammation. Non-limiting examples of such molecules are set out above. Such molecules are optionally attached via cleavable linkers designed to allow the molecules to be released at or near the target site.

By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶-chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium⁹⁹m and/or indium¹¹¹ are also often preferred due to their low energy and suitability for long range detection. Radioactively labeled monoclonal antibodies may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

B. Combination Therapies

It may also be desirable to provide combination treatments using antibodies of the present invention in conjunction with any of the foregoing therapeutic modalities. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the antibody and the other includes the other agent.

Alternatively, the antibody may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the MUC1 antagonist or the other therapy will be desired. Various combinations may be employed, where the antibody is “A,” and the other therapy is “B,” as exemplified below:

| | | | | | |
|---|---|---|---|---|---|
| A/B/A | B/A/B | B/B/A | A/A/B | B/A/A | A/B/B |
| B/B/B/A | B/B/A/B | A/A/B/B | A/B/A/B | A/B/B/A | B/B/A/A |
| B/A/B/A | B/A/A/B | B/B/B/A | A/A/A/B | B/A/A/A | A/B/A/A |
| A/A/B/A | A/B/B/B | B/A/B/B | B/B/A/B | | |

Other combinations are contemplated. To kill cells, inhibit cell growth, inhibit metastasis, inhibit angiogenesis or otherwise reverse or reduce the malignant phenotype of tumor cells, using the methods and compositions of the present invention, one may contact a target cell with an antibody and at least one other therapy. These therapies would be provided in a combined amount effective to kill or inhibit proliferation of the cell. This process may involve contacting the cells with the agents/therapies at the same time.

### V. Immunodetection Methods

In still further embodiments, there are immunodetection methods for binding, purifying, removing, quantifying and otherwise generally detecting MUC1 and its associated antigens. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of MUC1 antibodies also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample and contacting the sample with a first antibody in accordance with embodiments discussed herein, as the case may be, under conditions effective to allow the formation of immunocomplexes.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to MUC1 present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

1. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the invention are immobilized onto a selected surface exhibiting protein affinity, such as a well in a polystyrene microtiter plate. Then, a test composition suspected of containing the MUC1 is added to the wells. After binding and washing to remove non-specifically bound immune complexes, the bound antigen may be detected. Detection may be achieved by the addition of another anti-MUC1 antibody that is linked to a detectable label. This type of ELISA is a simple “sandwich ELISA.” Detection may also be achieved by the addition of a second anti-MUC1 antibody, followed by the addition of a third antibody that has binding affinity for the second antibody, with the third antibody being linked to a detectable label.

In another exemplary ELISA, the samples suspected of containing the MUC1 antigen are immobilized onto the well surface and then contacted with anti-MUC1 antibody. After binding and washing to remove non-specifically bound immune complexes, the bound anti-MUC1 antibodies are detected. Where the initial anti-MUC1 antibodies are linked to a detectable label, the immune complexes may be detected directly. Again, the immune complexes may be detected using a second antibody that has binding affinity for the first anti-MUC1 antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

2. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF, but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

3. Immunohistochemistry

The antibodies may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

4. Immunodetection Kits

In still further embodiments, there are immunodetection kits for use with the immunodetection methods described above. The immunodetection kits will thus comprise, in suitable container means, a first antibody that binds to MUC1 antigen, and optionally an immunodetection reagent.

In certain embodiments, the MUC1 antibody may be pre-bound to a solid support, such as a column matrix and/or well of a microtitre plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with embodiments discussed herein.

The kits may further comprise a suitably aliquoted composition of the MUC1 antigen, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits will also include a means for containing the antibody, antigen, and any other reagent containers in close confinement for commercial sale. Such containers may include injection or blow-molded plastic containers into which the desired vials are retained.

### VI. EXAMPLES

The following examples are included to demonstrate preferred embodiments. It should be appreciated by those of skill in the art that the techniques disclosed in the examples which follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Antibody Production and Screening

Immunization and Testing the Immune Sera.

Mice were immunized with the protein that contained the cytoplasmic domain of MUC1 fused to GST (GST-CD) as antigen. Two mice were immunized with the antigen mixed with Freund's complete adjuvant with repeated boosting as per the schedule shown in Table 1. Final boosting was done with 50 mg of antigen intravenously after checking the immune sera. Preimmune serum was collected to be used as negative control. Immune serum was collected after the 7^{th} injection as per the schedule and the serum was tested by both Western blotting and ELISA as per the methods described below.

TABLE 1
Immunization Schedule and Details
Mouse Strain: Swiss-webser, C57B1/6 Age: 6 weeks
Sex: Female Number of animals: 2

| Time | Dose/animal | Adjuvant | Comments |
|---|---|---|---|
| Day 1 | 100 μg | Complete | Pre-immunization bleed #1-N, |
| Day 4 | 100 μg | PBS | #2-N (black) on Day −3 |
| Day 6 | 50 μg | Incomplete | |
| Day 8 | 50 μg | PBS | 4th injection |
| Day 11 | 50 μg | Incomplete | 5th injection |
| Day 13 | 50 μg | PBS | 6th injection |
| Day 15 | 50 μg | Incomplete | 7th injection |
| Day 18 | 50 μg | PBS | Bleeding |
| Day 18 | | | 8th injection |
| Day 32 | 100 μg | Incomplete | 1st ext.injection i.p. |

ELISA was performed by coating the ELISA plates with 100 μl of 100 μg/ml GST-MUC1 (GST-CD) or GST-irrelevant protein. Hybridoma supernatants or immune sera were screened against the coated proteins by incubating with the plate for 1 hr. The bound antibody was detected by incubating with specific secondary antibody conjugated to HRP (horseradish peroxidase). In this case, the secondary antibody used was anti-mouse Ig-HRP (specific for light chain). Further, the reaction was developed with HRP-specific substrate.

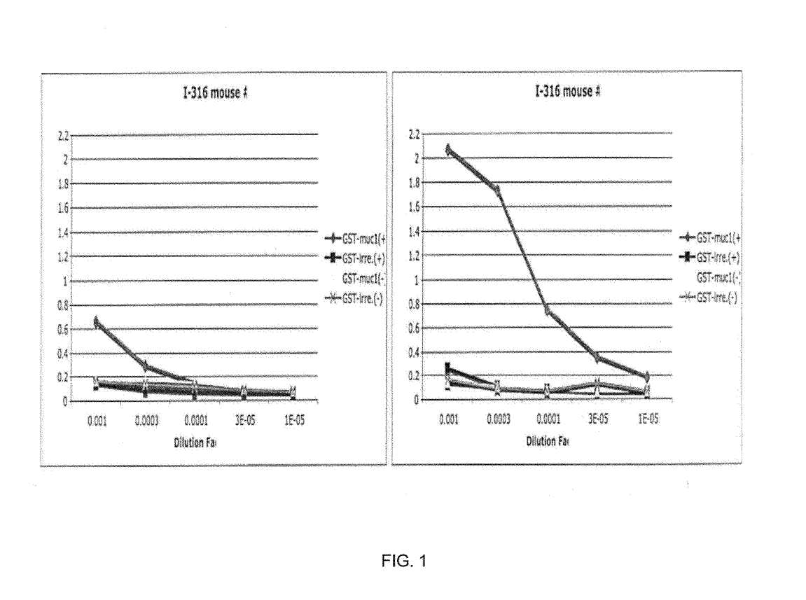

The result of the ELISA performed with the immune sera has been presented in Table 2 and graphically represented in FIG. 1. As seen in Table 2, testing serum from mouse 1 showed a detectable signal till 1:10,000 dilution where as the signal for the serum of mouse #2 exhibited a detectable signal even when it is diluted 1:100,000. However, the sera from both mice did not yield any significant signal with the GST-irrelevant protein. These results suggested that the spleen from mouse 2 is suitable for fusion and production of hybridoma.

TABLE 2
Testing the immune sera by ELISA

| | 1 | 2 | 3 | 4 | 5 | 6 | 7 | 8 | 9 | 10 | 11 | 12 |
|---|---|---|---|---|---|---|---|---|---|---|---|---|
| A | | | | | | | | | | | | |
| B | | | | | | | | | | | | |
| C | | | | | | | | | | | | |
| D | | | | | | | | | | | | |
| E* | | 1:1K | 1:3K | 1:10K | 1:30K | 1:100K | 1:1K | 1:3K | 1:10K | 1:30K | 1:100K | (+) |
| F* | (++) | 1:1K | 1:3K | 1:10K | 1:30K | 1:100K | 1:1K | 1:3K | 1:10K | 1:30K | 1:100K | (+) |
| G** | (++) | 1:1K | 1:3K | 1:10K | 1:30K | 1:100K | 1:1K | 1:3K | 1:10K | 1:30K | 1:100K | (−) |
| H** | | 1:1K | 1:3K | 1:10K | 1:30K | 1:100K | 1:1K | 1:3K | 1:10K | 1:30K | 1:100K | (−) |
| E* | 0.063 | 0.174 | 0.167 | 0.151 | 0.104 | 0.078 | 0.67 | 0.303 | 0.147 | 0.085 | 0.07 | 0.566 |
| F* | 2.145 | 0.165 | 0.138 | 0.117 | 0.087 | 0.072 | 0.149 | 0.094 | 0.074 | 0.064 | 0.064 | 0.293 |
| G** | 2.208 | 0.143 | 0.109 | 0.078 | 0.065 | 0.067 | 2.082 | 1.742 | 0.759 | 0.363 | 0.192 | 0.087 |
| H** | 0.07 | 0.167 | 0.093 | 0.07 | 0.136 | 0.064 | 0.268 | 0.109 | 0.08 | 0.064 | 0.063 | 0.065 |

Coating with GST-muc1 protein (100 ng/100 μl well in PBS)
Coating with GST-irrelevant protein (100 ng/100 μl well in PBS)
*mouse #1
**mouse #2
(++)—DG122 anti-GST Ab sup neat
(+)—I-205 mFc-Muc1 serum #4 1:1K
(−)—Sp2/O sup

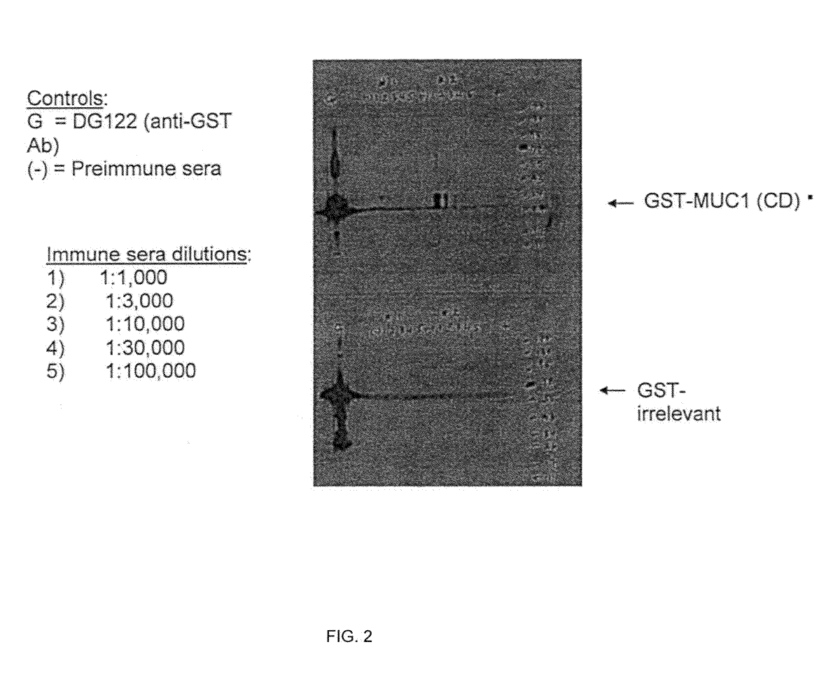

Further analysis was performed by testing both antisera in Western blot against 20 μg of GST-MUC1 (CD) and GST-irrelevant protein ran on a gel and transferred to nitrocellulose/nylon membrane. The results are presented in FIG. 2. As shown in FIG. 2, antiserum from mouse 1 detected MUC1 (CD) from the gel at 1:3000 dilution and antiserum 2 detected MUC1 (CD) at 1:10,000 dilution. These results confirm the results obtained by ELISA and indicated that mouse #2 had a better response for the antigen.

Fusion for Hybridoma and Primary Screening.

Based on the results obtained from the ELISA and Western blot, spleen from mouse #2 was used for fusion with myeloma cells to generate hybridoma. Spleen-myeloma fusion was performed by mixing mouse myeloma cells sp2/0-Ag14 and splenocytes from mouse #2 in 1:3 ratio in the presence of polyethylene glycol (PEG). Post-fusion cell culture was carried out in selective HAT medium. Hybridomas selected by indirect ELISA or Western blot using recombinant MUC1-CD as the antigen were subcloned to stability by limiting dilution protocol.

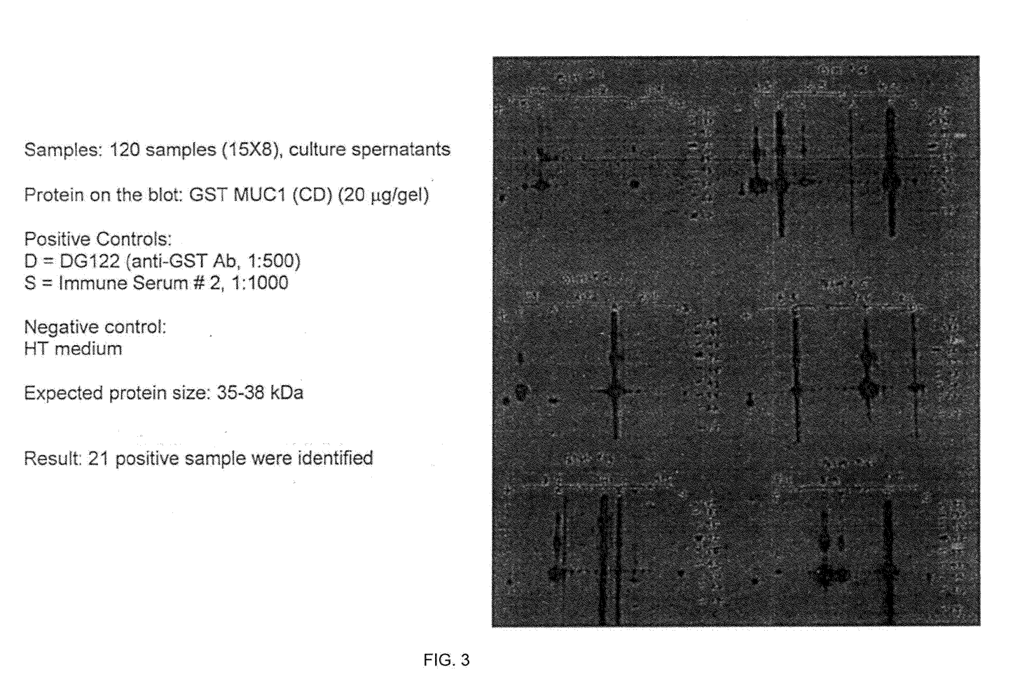

The fused cells were plated in 120 wells (15×8 plates). After sufficient growth of cells, the supernatant from each well was used for screening the samples in Western blot as mentioned above. 120 samples were screened against 20 μg of GST-MUC1 (CD) protein on the gel. Screening of the supernatants from these samples identified 21 positive samples as they yielded the expected protein band of 35-38 kDa size (see FIG. 3). All these 21 positive clones were carried over for further processing.

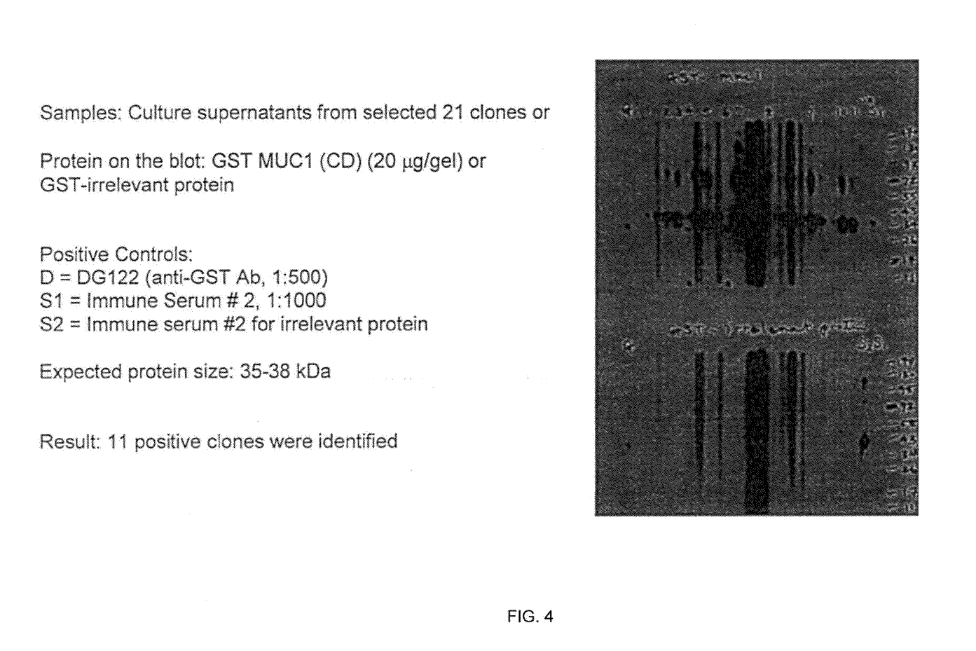

The 21 primary parental clones identified as GST-MUC1 (CD)-reactive positive clones were transferred to 24-well plates. After sufficient growth of cells, the supernatant was collected from these 21 clones and subjected to further screening by Western blot as mentioned above. The results of these screening by Western blot has been presented in FIG. 4. As shown in FIG. 4, 11 of the 21 clones were identified as positive clones as they reacted to the GST-MUC1 (CD) protein on the blotting membrane and highlighted a band of 35-38 kDa. List of the positive clones were presented in Table 3.

TABLE 3
GST-MUC1 (CD) Reactive Clones

| Serial number | Name of clones |
|---|---|
| 1 | 316.2.1B6 |
| 2 | 316.2.1H8 |
| 3 | 316.2.1E9 |
| 4 | 316.2.2A8 |
| 5 | 316.2.3E7 |
| 6 | 316.2.3G10 |
| 7 | 316.2.4E5 |
| 8 | 316.2.5C3 |
| 9 | 316.2.7F6 |
| 10 | 316.2.8D9 |
| 11 | 316.2.8F9 |

Clones that were positively reacting to GST-MUC1 (CD) were tested by an alternative method (ELISA). An ELISA was performed by coating His-MUC1 (CD) either at 25 ng or 100 ng/ml. 100 ml of test supernatants were incubated for an hour and the bound antibody from the supernatant was revealed by incubating with anti-mouse Ig conjugated to HRP (1:500 dilution) and subsequent development of the HRP reaction with it's substrate. The results and plate map were shown in Table 4. The upper part of the table shows the plate map, which indicates the location of the test supernatants, and the lower part shows the corresponding absorbance values for those supernatants. As indicated in the table, 9 of the 11 parental clones tested were positive for the reactivity against His-MUC1 (CD). However, clone 1B6 had displayed a low level absorbance suggesting that may not be a preferred clone. Based on this result, 4 of the high expression clones (3E7, 4E5, 7F6 and 8D9) were chosen for further subcloning and screening.

TABLE 4
Plate Map and ELISA Results of Culture Supernatants from Selected Clones

| | 25 ng/ml His-MUC1 (CD) | | | | | | 100 ng/ml His-MUC1 (CD | | | | | |
|---|---|---|---|---|---|---|---|---|---|---|---|---|
| | 1 | 2 | 3 | 4 | 5 | 6 | 7 | 8 | 9 | 10 | 11 | 12 |
| A | Medium | Medium | Medium | 5C3 | 5C3 | 5C3 | Medium | Medium | Medium | 5C3 | 5C3 | 5C3 |
| B | 1B6 | 1B6 | 1B6 | 7F6 | 7F6 | 7F6 | 1B6 | 1B6 | 1B6 | 7F6 | 7F6 | 7F6 |
| C | 1H8 | 1H8 | 1H8 | 8D9 | 8D9 | 8D9 | 1H8 | 1H8 | 1H8 | 8D9 | 8D9 | 8D9 |
| D | 1E9 | 1E9 | 1E9 | 8F9 | 8F9 | 8F9 | 1E9 | 1E9 | 1E9 | 8F9 | 8F9 | 8F9 |
| E | 2A8 | 2A8 | 2A8 | | | | 2A8 | 2A8 | 2A8 | | | |
| F | 3E7 | 3E7 | 3E7 | | | | 3E7 | 3E7 | 3E7 | | | |
| G | 3G10 | 3G10 | 3G10 | | | | 3G10 | 3G10 | 3G10 | | | |
| H | 4E5 | 4E5 | 4E5 | | | | 4E5 | 4E5 | 4E5 | | | |
| A | 0.056 | 0.048 | 0.047 | 1.289 | 1.538 | 1.6 | 0.048 | 0.048 | 0.049 | 2.42 | 2.322 | 2.111 |
| B | 0.19 | 0.192 | 0.2 | 1.7 | 1.997 | 1.943 | 0.49 | 0.479 | 0.446 | 2.742 | 2.771 | 2.208 |
| C | 0.92 | 0.994 | 0.919 | 1.791 | 1.851 | 1.841 | 1.97 | 1.8 | 1.749 | 2.736 | 2.675 | 2.402 |
| D | 1.249 | 1.318 | 1.235 | 1.203 | 1.364 | 1.407 | 2.519 | 2.368 | 2.464 | 2.345 | 2.532 | 2.003 |
| E | 0.051 | 0.05 | 0.053 | 0.046 | 0.047 | 0.046 | 0.056 | 0.056 | 0.056 | 0.047 | 0.048 | 0.047 |
| F | 1.747 | 1.6 | 1.523 | 0.046 | 0.046 | 0.046 | 2.574 | 2.648 | 2.67 | 0.046 | 0.046 | 0.046 |
| G | 0.059 | 0.06 | 0.059 | 0.049 | 0.051 | 0.046 | 0.071 | 0.07 | 0.071 | 0.046 | 0.046 | 0.047 |
| H | 1.999 | 1.974 | 1.896 | 0.046 | 0.048 | 0.048 | 2.615 | 2.629 | 1.167 | 0.046 | 0.047 | 0.047 |

Secondary Screening.

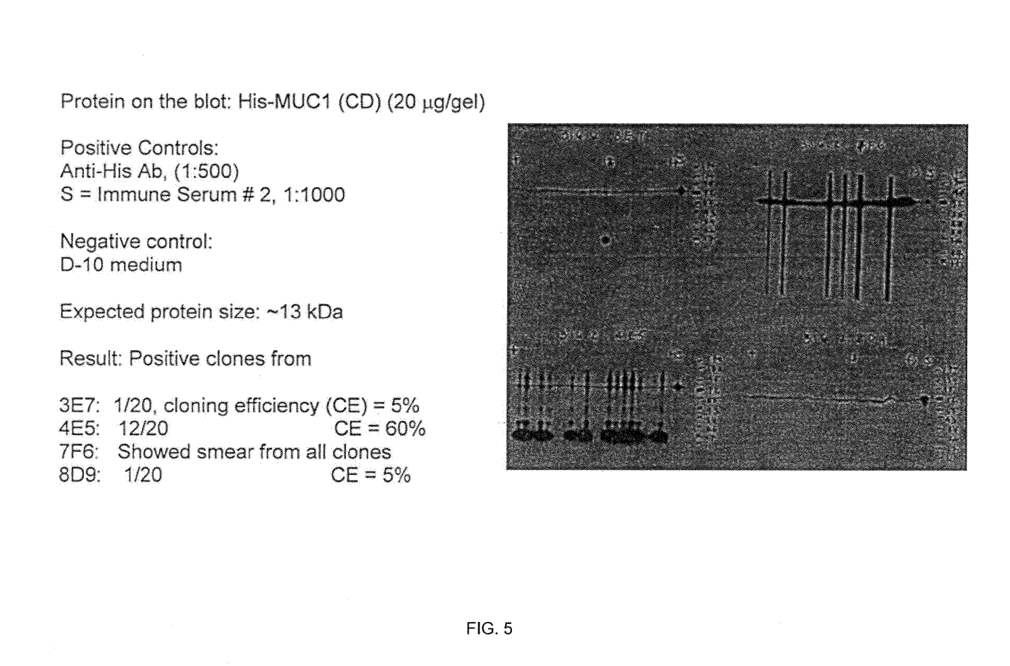

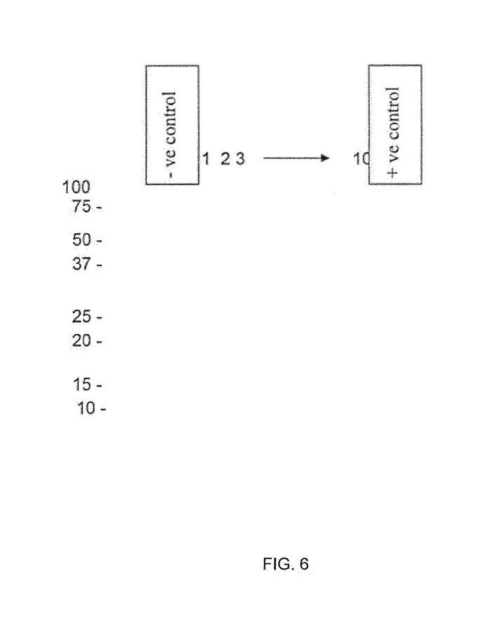

The selected clones were subjected to subcloning and the subclone supernatants were tested by Western blot. For each parental clone, 20 subclones were obtained. Out of 20 subclones, some were single clones and some of them had more than one clone in the well. The composition of the subclones is as follows:
3E7: #1-#11 single colony/well and #12-20 > one colony/well
4E5: #1-#18 single colony/well and #19-20 > one colony/well
7F6: #1-#19 single colony/well and #20 > one colony/well
8D9: #1-#19 single colony/well and #20 > one colony/well
   FIG. 5 shows the results of analysis of the subclones by Western blot. Western blot analysis of the subclones yielded 1/20 positive clone from 3E7 parental, 12/20 positive from 4E5 parental and 1/20 positive from 8D9 parental with the cloning efficiency of 5%, 60% and 5% respectively. Subclones from 7F6 yielded only a smear in the analysis. Since, the cloning efficiency was <80%, a second round of subclone screening was initiated.

In the second round of subcloning, the cloning efficiency was improved to 70% and 95% respectively for 3E7 and 4E5 and no positive clone was identified from 8D9. Therefore, the subcloning was repeated for 8D9 and 2 positive clones were identified at this time. 8D9 subcloning was continued with different subclones of 8D9 until enough number of tertiary subclones were obtained.

Based on the results of subcloning, it was decided to pursue with 3 subclones from each parental clone. The chosen clones are as follows:
(1) 3E7.D11.C1
(2) 3E7.D11.C10
(3) 3E7.D11.H9
(4) 4E5.H8.F1
(5) 4E5.H8.F11
(6) 4E5.H8.G2
(7) 8D9.2E11.E4
(8) 8D9.2E11.D12
(9) 8D9.2E11.B7

### Example 2

### Antibody Characterization

Selection of Final Clones and Epitope Mapping.

Since, the antigen used was the whole cytoplasmic domain (72 amino acids) of MUC1 [GST-MUC1 (CD)], it was anticipated that there may be different antibodies directed against different epitopes of the protein. In order to test this possibility, different fragments of MUC1-CD have been generated and used as the coating antigen in ELISA and the results are presented below.

Initially, culture supernatants of subclones from 2 of the parental clones (4E5 and 8D9) were tested by ELISA by coating the ELISA plate with 500 ng/ml of GST-CD (1-45) and/or GST-CD (46-72). ELISA was performed with the standard procedure as mentioned above. The results obtained from the assay along with the plate map has been presented in Table 5. As shown in the table, supernatants from all the subclones effectively reacted to GST-CD (1-45) and the reactivity with the fragment GST-CD (46-72) was low but more than the negative control. These results suggest that the epitope for the antibody is present within amino acids 1-45. The low level reactivity may be due to an overlapping epitope present around amino acid 46 or there may be a cross reacting epitope present in the fragment 46-72. Supernatants of the subclones (obtained from the parental clone 3E7) were also tested similarly by ELISA against both the fragments of MUC1-CD (1-45 and 46-72). Subclones obtained from 3E7 also had a similar reactivity to the MUC1-CD fragments as shown in Table 6.

TABLE 5
Plate Map and ELISA Results of Culture Supernatants from the Selected Subclones of 4E5 and 8D9

| | 500 ng/ml GST-MUC1 (CD) (1-45) | | | | | | 500 ng/ml GST-MUC1 (CD) (46-72) | | | | | |
|---|---|---|---|---|---|---|---|---|---|---|---|---|
| | 1 | 2 | 3 | 4 | 5 | 6 | 7 | 8 | 9 | 10 | 11 | 12 |
| A | Medium | Medium | Positive | Positive | | | Medium | Medium | Positive | Positive | | |
| B | 4E5-parental | 4E5-parental | | | | | 4E5-parental | 4E5-parental | | | | |
| C | 4E5-H8.G2 | 4E5-H8.G2 | | | | | 4E5-H8.G2 | 4E5-H8.G2 | | | | |
| D | 4E5-H8.F1 | 4E5-H8.F1 | | | | | 4E5-H8.F1 | 4E5-H8.F1 | | | | |
| E | 4E5-H8.F11 | 4E5-H8.F11 | | | | | 4E5-H8.F11 | 4E5-H8.F11 | | | | |
| F | 8D9.2E11.E4 | 8D9.2E11.E4 | | | | | 8D9.2E11.E4 | 8D9.2E11.E4 | | | | |
| G | 8D9.2E11.D12 | 8D9.2E11.D12 | | | | | 8D9.2E11.D12 | 8D9.2E11.D12 | | | | |
| H | 8D9.2E11.B7 | 8D9.2E11.B7 | | | | | 8D9.2E11.B7 | 8D9.2E11.B7 | | | | |
| A | 0.096 | 0.046 | 2.858 | 2.783 | 0.045 | 0.044 | 0.052 | 0.052 | 0.151 | 0.146 | 0.048 | 0.041 |
| B | 2.887 | 2.895 | | | | | 0.28 | 0.279 | | | | |
| C | 2.935 | 2.96 | | | | | 0.336 | 0.331 | | | | |
| D | 2.888 | 2.934 | | | | | 0.323 | 0.307 | | | | |
| E | 2.855 | 2.913 | | | | | 0.259 | 0.291 | | | | |
| F | 2.878 | 2.94 | | | | | 0.235 | 0.235 | | | | |
| G | 2.866 | 2.778 | | | | | 0.252 | 0.24 | | | | |
| H | 2.7 | 2.818 | | | | | 0.26 | 0.24 | | | | |

Positive = supernatant from one of the parental clones from the primary screening used as a +ve control.

TABLE 6
Plate Map and ELISA Results of Culture Supernatants from the Subclones of 3E7

| 500 ng/ml GST-MUC1 (CD) (1-45) | | | | | | 500 ng/ml GST-MUC1 (CD) (46-72) | | | | | |
|---|---|---|---|---|---|---|---|---|---|---|---|
| 1 | 2 | 3 | 4 | 5 | 6 | 7 | 8 | 9 | 10 | 11 | 12 |

| | | | | |
|---|---|---|---|---|
| A | Medium | Medium | Medium | Medium |
| B | 3E7.D11.H9.H3.D1 | 3E7.D11.H9.H3.D1 | 3E7.D11.H9.H3.D1 | 3E7.D11.H9.H3.D1 |
| C | 3E7.D11.H9.H3..E2 | 3E7.D11.H9.H3..E2 | 3E7.D11.H9.H3..E2 | 3E7.D11.H9.H3..E2 |
| D | 3E7.D11.H9.H3.D3 | 3E7.D11.H9.H3.D3 | 3E7.D11.H9.H3.D3 | 3E7.D11.H9.H3.D3 |
| E | Positive | Positive | Positive | Positive |
| F | | | | |
| G | | | | |
| H | | | | |
| A | 0.05 | 0.05 | 0.058 | 0.057 |
| B | 2.8 | 2.714 | 0.358 | 0.347 |
| C | 2.747 | 2.748 | 0.362 | 0.357 |
| D | 2.79 | 2.754 | 0.349 | 0.356 |
| E | 2.67 | 2.646 | 0.2 | 0.173 |
| F | 0.051 | 0.05 | 0.067 | 0.067 |
| G | 0.051 | 0.05 | 0.066 | 0.061 |
| H | 0.051 | 0.05 | 0.061 | 0.066 |

Positive = supernatant from one of the parental clones from the primary screening used as a +ve control

Further, to narrow down the range of the epitope, an ELISA was performed by coating 500 ng/ml of various fragments of GST-CD generated with amino acids (1-45), (20-72), (25-72), (30-72), (35-72) and (40-72). Nine supernatants from the subclones of 3 parental clones and a parental supernatant were tested in this assay as shown in the plate map of Table 7a and Table 7b.

The data presented in Table 7a and Table 7b indicate that all the culture supernatants reacted only with GST-CD (1-45) that was used as positive control. However, none of the supernatants reacted to any of the fragments that had amino acid 20 and above. These results suggest that the epitope for all the subclones or parental clones exists between amino acids 1-20. In addition, the highly reactive part of the protein (CQCRRKN) (amino acid 1-7) was mutated and the ability to bind the antibodies was tested in ELISA. As shown Table 8a and 8b, none of the mutations in the CQC portion of the protein affected its binding to the antibody as the absorbance values obtained were very high. GST-CD (6-21) was also used along with them for coating and it yielded a high absorbance. These results suggest that the epitope may not be present within the first few amino acids of MUC1-CD. Therefore, it was decided to use protein fragments or peptides of MUC1-CD which contains amino acids 1-20 as the coating antigen (binding antigen). Accordingly, GO-203-2 (a 7-mer peptide with amino acids 1-7) and it is mutated version GO-203-AQA were used as coating antigen along with the positive control fragment MUC1-CD (1-45). The supernatants used and the absorbance values obtained in the ELISA are shown in Table 9. All the hybridoma supernatants used reacted only with GST-CD (1-45) and not with the indicated peptides (amino acid 1-7). The absorbance value 0.733 and 0.408 obtained in wells G5 and H5 are due to accidental spillage of solution from the positive wells and not due to reactivity with the coated peptides. It is evident from the values of the duplicate wells. These results indicate that the epitope may be present between amino acids 8-19 or the antibodies may not have reacted to the peptide because of its dextrorotatory conformation. To verify this fact, an ELISA was performed by coating the plate with 100 ml (250 ng/ml and 500 ng/ml) of GO-201 (a 15-mer peptide with levorotatory conformation, amino acid 1-15). Plate was also coated with GST-CD (1-45) as positive control for coating. One representative subclone from each parental group was tested in this ELISA along with the culture medium as negative control. The result of this assay has been presented in Table 10. All the three subclones reacted with the peptide (GO-201) both at 250 ng/ml and 500 ng/ml concentration. These results when compared with the results mentioned in Tables 8a, 8b and 9 clearly indicate that the epitope for these test antibodies are located between amino acid residues 8-15.

TABLE 7a
Plate map and ELISA results of culture supernatants tested against various fragments of MUC1-CD (Plate 1)

| | 500 ng/ml of GST-CD (1-45) | | | | 500 ng/ml of GST-CD (20-72) | | | | 500 ng/ml of GST-CD (25-72) | | | |
|---|---|---|---|---|---|---|---|---|---|---|---|---|
| | 1 | 2 | 3 | 4 | 5 | 6 | 7 | 8 | 9 | 10 | 11 | 12 |
| A | Medium | | 3E7.D11.H9.H3.D1 | | Medium | | 3E7.D11.H9.H3.D1 | | Medium | | 3E7.D11.H9.H3.D1 | |
| B | 4E5-parental | | 3E7.D11.H9.H3.D3 | | 4E5-parental | | 3E7.D11.H9.H3.D3 | | 4E5-parental | | 3E7.D11.H9.H3.D3 | |
| C | 4E5-H8.G2 | | 3E7.D11.H9.H3.E2 | | 4E5-H8.G2 | | 3E7.D11.H9.H3.E2 | | 4E5-H8.G2 | | 3E7.D11.H9.H3.E2 | |
| D | 4E5-H8.F1 | | −ve control | | 4E5-H8.F1 | | −ve control | | 4E5-H8.F1 | | −ve control | |
| E | 4E5-H8.F11 | | | | 4E5-H8.F11 | | | | 4E5-H8.F11 | | | |
| F | 8D9.2E11.E4 | | | | 8D9.2E11.E4 | | | | 8D9.2E11.E4 | | | |
| G | 8D9.2E11.D12 | | | | 8D9.2E11.D12 | | | | 8D9.2E11.D12 | | | |
| H | 8D9.2E11.B7 | | | | 8D9.2E11.B7 | | | | 8D9.2E11.B7 | | | |
| A | 2.713 | 2.777 | 2.912 | 2.975 | 0.063 | 0.063 | 0.058 | 0.056 | 0.054 | 0.057 | 0.054 | 0.052 |
| B | 2.814 | 2.899 | 2.917 | 2.911 | 0.064 | 0.68 | 0.055 | 0.057 | 0.054 | 0.062 | 0.053 | 0.059 |
| C | 2.756 | 2.884 | 0.051 | 0.56 | 0.071 | 0.068 | 0.049 | 0.051 | 0.063 | 0.060 | 0.048 | 0.54 |
| D | 2.803 | 2.897 | | | 0.058 | 0.065 | | | 0.055 | 0.053 | | |
| E | 2.791 | 2.808 | | | 0.053 | 0.056 | | | 0.060 | 0.053 | | |
| F | 2.921 | 2.820 | | | 0.053 | 0.053 | | | 0.055 | 0.052 | | |
| G | 2.804 | 2.875 | | | 0.054 | 0.055 | | | 0.056 | 0.054 | | |
| H | 2.834 | 2.844 | | | 0.058 | 0.058 | | | 0.056 | 0.056 | | |

TABLE 7b
Plate map and ELISA results of culture supernatants tested against various fragments of MUC1-CD (Plate 2)

| | 500 ng/ml of GST-CD (30-72) | | | | 500 ng/ml of GST-CD (35-72) | | | | 500 ng/ml of GST-CD (40-72) | | | |
|---|---|---|---|---|---|---|---|---|---|---|---|---|
| | 1 | 2 | 3 | 4 | 5 | 6 | 7 | 8 | 9 | 10 | 11 | 12 |
| A | Medium | | 3E7.D11.H9.H3.D1 | | Medium | | 3E7.D11.H9.H3.D1 | | Medium | | 3E7.D11.H9.H3.D1 | |
| B | 4E5-parental | | 3E7.D11.H9.H3.D3 | | 4E5-parental | | 3E7.D11.H9.H3.D3 | | 4E5-parental | | 3E7.D11.H9.H3.D3 | |
| C | 4E5-H8.G2 | | 3E7.D11.H9.H3.E2 | | 4E5-H8.G2 | | 3E7.D11.H9.H3.E2 | | 4E5-H8.G2 | | 3E7.D11.H9.H3.E2 | |
| D | 4E5-H8.F1 | | −ve control | | 4E5-H8.F1 | | −ve control | | 4E5-H8.F1 | | −ve control | |
| E | 4E5-H8.F11 | | | | 4E5-H8.F11 | | | | 4E5-H8.F11 | | | |
| F | 8D9.2E11.E4 | | | | 8D9.2E11.E4 | | | | 8D9.2E11.E4 | | | |
| G | 8D9.2E11.D12 | | | | 8D9.2E11.D12 | | | | 8D9.2E11.D12 | | | |
| H | 8D9.2E11.B7 | | | | 8D9.2E11.B7 | | | | 8D9.2E11.B7 | | | |
| A | 0.064 | 0.057 | 0.057 | 0.058 | 0.130 | 0.059 | 0.053 | 0.053 | 0.052 | 0.055 | 0.054 | 0.053 |
| B | 0.057 | 0.055 | 0.079 | 0.057 | 0.085 | 0.054 | 0.053 | 0.053 | 0.059 | 0.056 | 0.054 | 0.051 |
| C | 0.055 | 0.054 | 0.124 | 0.053 | 0.053 | 0.197 | 0.073 | 0.049 | 0.050 | 0.055 | 0.053 | 0.050 |
| D | 0.053 | 0.053 | | | 0.052 | 0.056 | | | 0.050 | 0.051 | | |
| E | 0.053 | 0.053 | | | 0.053 | 0.052 | | | 0.057 | 0.053 | | |
| F | 0.053 | 0.054 | | | 0.051 | 0.051 | | | 0.055 | 0.051 | | |
| G | 0.054 | 0.054 | | | 0.053 | 0.053 | | | 0.054 | 0.052 | | |
| H | 0.056 | 0.055 | | | 0.053 | 0.055 | | | 0.054 | 0.054 | | |

TABLE 8a
ELISA for epitope mapping with the hybridoma subclones

| Coating Ag | | | | | | | | | | | |
|---|---|---|---|---|---|---|---|---|---|---|---|
| GST-CD (1-45) | | | | GST-CD (6-21) | | | | GST-CD (AQA) | | | |
| 1 | 2 | 3 | 4 | 5 | 6 | 7 | 8 | 9 | 10 | 11 | 12 |

| A | Medium | | 3E7.D11.H9.H3.D1 | | Medium | | Medium | | 3E7.D11.H9.H3.D1 | |
|---|---|---|---|---|---|---|---|---|---|---|
| B | Medium | | 3E7.D11.H9.H3.D3 | | 4E5-H8.F1 | | Medium | | 3E7.D11.H9.H3.D3 | |
| C | 4E5-H8.G2 | | 3E7.D11.H9.H3.E2 | | 3E7.D11.H9.H3.E2 | | 4E5-H8.G2 | | 3E7.D11.H9.H3.E2 | |
| D | 4E5-H8.F1 | | Positive | | 8D9.2E11.B7 | | 4E5-H8.F1 | | Medium | |
| E | 4E5-H8.F11 | | | | 3E7.D11.H9.H3.D3 | | 4E5-H8.F11 | | | |
| F | 8D9.2E11.E4 | | | | | | 8D9.2E11.E4 | | | |
| G | 8D9.2E11.D12 | | | | | | 8D9.2E11.D12 | | | |
| H | 8D9.2E11.B7 | | | | | | 8D9.2E11.B7 | | | |
| A | 0.048 | 0.046 | 2.89 | 2.977 | 0.431 | 0.052 | 0.063 | 0.057 | 3.329 | 3.428 |
| B | 0.048 | 0.047 | 2.865 | 2.89 | 2.425 | 2.506 | 0.126 | 0.081 | 3.24 | 3.214 |
| C | 3.054 | 2.936 | 2.634 | 2.793 | 2.559 | 2.425 | 2.963 | 2.926 | 3.206 | 3.215 |
| D | 3.12 | 2.852 | 1.881 | 2.137 | 1.48 | 1.591 | 2.876 | 3.02 | 0.065 | 0.067 |
| E | 3.107 | 2.841 | | | 2.01 | 1.792 | 2.982 | 3.147 | | |
| F | 3.092 | 2.887 | | | | | 3.26 | 3.283 | | |
| G | 2.976 | 2.673 | | | | | 3.025 | 3.065 | | |
| H | 2.9 | 2.925 | | | | | 3.087 | 3.136 | | |

Plate was coated with 500 ng/ml of GST-CD (6-21), GST-CD (AQA), GST-CD (AQC) and GST-CD (CQA).
GST-CD (1-45) was used as positive control for coating antigen.
Supernatant from one of the parental clones was used as positive control antibody.
Hybridoma culture medium was used as negative control.

TABLE 8b
ELISA for epitope mapping with the hybridoma subclones

| Coating Ag | | | | | | | | | | | |
|---|---|---|---|---|---|---|---|---|---|---|---|
| GST-CD (AQC) | | | | | | GST-CD (CQA) | | | | | |
| 1 | 2 | 3 | 4 | 5 | 6 | 7 | 8 | 9 | 10 | 11 | 12 |

| A | Medium | | 3E7.D11.H9.H3.D1 | | Medium | | 3E7.D11.H9.H3.D1 | |
|---|---|---|---|---|---|---|---|---|
| B | 4E5-parental | | 3E7.D11.H9.H3.D3 | | 4E5-parental | | 3E7.D11.H9.H3.D3 | |
| C | 4E5-H8.G2 | | 3E7.D11.H9.H3.E2 | | 4E5-H8.G2 | | 3E7.D11.H9.H3.E2 | |
| D | 4E5-H8.F1 | | Positive | | 4E5-H8.F1 | | Positive | |
| E | 4E5-H8.F11 | | | | 4E5-H8.F11 | | | |
| F | 8D9.2E11.E4 | | | | 8D9.2E11.E4 | | | |
| G | 8D9.2E11.D12 | | | | 8D9.2E11.D12 | | | |
| H | 8D9.2E11.B7 | | | | 8D9.2E11.B7 | | | |
| A | 0.053 | 0.05 | 3.105 | 3.015 | 0.051 | 0.05 | 3.163 | 3.13 |
| B | 3.249 | 2.923 | 2.95 | 3.011 | 2.902 | 2.643 | 2.645 | 2.791 |
| C | 3.258 | 3.085 | 2.725 | 2.792 | 2.69 | 3.159 | 2.744 | 2.96 |
| D | 2.98 | 2.928 | 2.946 | 2.835 | 2.985 | 2.775 | 3.036 | 2.841 |
| E | 3.235 | 2.893 | | | 2.738 | 2.717 | | |
| F | 3.002 | 2.959 | | | 2.656 | 2.673 | | |
| G | 3.163 | 3.001 | | | 2.972 | 2.956 | | |
| H | 3.014 | 3.068 | | | 3.05 | 2.883 | | |

TABLE 9
ELISA for epitope mapping with the hybridoma subclones

| Coating Ag | | | | | | | | | | | |
|---|---|---|---|---|---|---|---|---|---|---|---|
| GO-203-2 | | | | GO-203-AQA | | | | GST-CD (1-45) | | | |
| 1 | 2 | 3 | 4 | 5 | 6 | 7 | 8 | 9 | 10 | 11 | 12 |

| A | Medium | | 3E7.D11.H9.H3.D1 | | Medium | | 3E7.D11.H9.H3.D1 | | Medium | |
|---|---|---|---|---|---|---|---|---|---|---|
| B | 4E5-parental | | 3E7.D11.H9.H3.D3 | | 4E5-parental | | 3E7.D11.H9.H3.D3 | | 4E5-H8.G2 | |
| C | 4E5-H8.G2 | | 3E7.D11.H9.H3.E2 | | 4E5-H8.G2 | | 3E7.D11.H9.H3.E2 | | Positive | |
| D | 4E5-H8.F1 | | Positive | | 4E5-H8.F1 | | Positive | | | |
| E | 4E5-H8.F11 | | | | 4E5-H8.F11 | | | | | |
| F | 8D9.2E11.E4 | | | | 8D9.2E11.E4 | | | | | |
| G | 8D9.2E11.D12 | | | | 8D9.2E11.D12 | | | | | |
| H | 8D9.2E11.B7 | | | | 8D9.2E11.B7 | | | | | |
| A | 0.047 | 0.046 | 0.052 | 0.052 | 0.046 | 0.048 | 0.052 | 0.055 | 0.138 | 0.058 |
| B | 0.053 | 0.052 | 0.053 | 0.052 | 0.053 | 0.054 | 0.055 | 0.054 | 1.852 | 1.619 |
| C | 0.053 | 0.056 | 0.052 | 0.053 | 0.056 | 0.055 | 0.207 | 0.054 | 1.388 | 1.183 |
| D | 0.054 | 0.055 | 0.046 | 0.047 | 0.057 | 0.058 | 0.064 | 0.049 | | |
| E | 0.051 | 0.053 | | | 0.065 | 0.055 | | | | |
| F | 0.050 | 0.051 | | | 0.056 | 0.054 | | | | |
| G | 0.052 | 0.052 | | | 0.733 | 0.058 | | | | |
| H | 0.051 | 0.066 | | | 0.408 | 0.055 | | | | |

Plate was coated with 250 ng/ml of GO-203-2 (amino acid 1-15), 500 ng/ml of GO-203-AQA (mutated version) and GST-CD (1-45) as positive control for coating antigen.
Supernatant from one of the parental clones was used as positive control antibody.
Hybridoma culture medium was used as negative control.

TABLE 10
ELISA for epitope mapping with the hybridoma subclones

| Coating Ag | | | | | | | | | | | |
|---|---|---|---|---|---|---|---|---|---|---|---|
| | | | GO-201 | | | GO-201 | | | | | |
| GST-CD (1-45) | | | (250 ng/ml) | | | (500 ng/ml) | | | | | |
| 1 | 2 | 3 | 4 | 5 | 6 | 7 | 8 | 9 | 10 | 11 | 12 |

| A | Medium | | | Medium | | | Medium | |
|---|---|---|---|---|---|---|---|---|
| B | 4E5.H8.F1 | | | 4E5.H8.F1 | | | 4E5.H8.F1 | |
| C | 8D9.2E11.D12 | | | 8D9.2E11.D12 | | | 8D9.2E11.D12 | |
| D | 3E7.D11.H9.H3.E2 | | | 3E7.D11.H9.H3.E2 | | | 3E7.D11.H9.H3.E2 | |
| E | | | | | | | | |
| F | | | | | | | | |
| G | | | | | | | | |
| H | | | | | | | | |
| A | 0.048 | 0.047 | 0.052 | 0.05 | 0.053 | 0.05 | 0.046 | 0.047 |
| B | 2.322 | 2.306 | 0.054 | 3.066 | 3.057 | 0.052 | 2.86 | 3.149 |
| C | 2.288 | 2.408 | 0.052 | 1.827 | 2.03 | 0.052 | 2.32 | 2.465 |
| D | 2.044 | 2.385 | 0.05 | 2.771 | 2.969 | 0.051 | 2.977 | 2.931 |
| E | | | | | | | | |
| F | | | | | | | | |
| G | | | | | | | | |
| H | | | | | | | | |

This observation was further confirmed by using a shorter version of the peptide GO-202 (7-mer peptide containing amino acid residues 1-7) along with positive control coating antigen GST-CD (1-45). In this assay, only three representative clones were used. All the supernatants tested yielded no reactivity to the coated peptide GO-202, whereas they reacted with the positive control coating antigen (Table 11), confirming that that the epitope is present between 8-15 of the protein.

TABLE 11
ELISA for epitope mapping with the hybridoma subclones

| Coating Ag | | | | | | | | | | | |
|---|---|---|---|---|---|---|---|---|---|---|---|
| | | | GO-202 | | | | | | | | |
| GST-CD (1-45) | | | (500 ng/ml) | | | | | | | | |
| 1 | 2 | 3 | 4 | 5 | 6 | 7 | 8 | 9 | 10 | 11 | 12 |

| A | Medium | | | Medium | |
|---|---|---|---|---|---|
| B | 4E5.H8.F1 | | | 4E5.H8.F1 | |
| C | 8D9.2E11.D12 | | | 8D9.2E11.D12 | |
| D | 3E7.D11.H9.H3.E2 | | | 3E7.D11.H9.H3.E2 | |
| E | | | | | |
| F | | | | | |
| G | | | | | |
| H | | | | | |
| A | 0.057 | 0.049 | 0.051 | 0.047 | 0.051 |
| B | 2.104 | 2.064 | 0.044 | 0.06 | 0.058 |
| C | 2.223 | 2.23 | 0.052 | 0.062 | 0.068 |
| D | 1.916 | 1.929 | 0.05 | 0.054 | 0.059 |
| E | | | | | |
| F | | | | | |
| G | | | | | |
| H | | | | | |

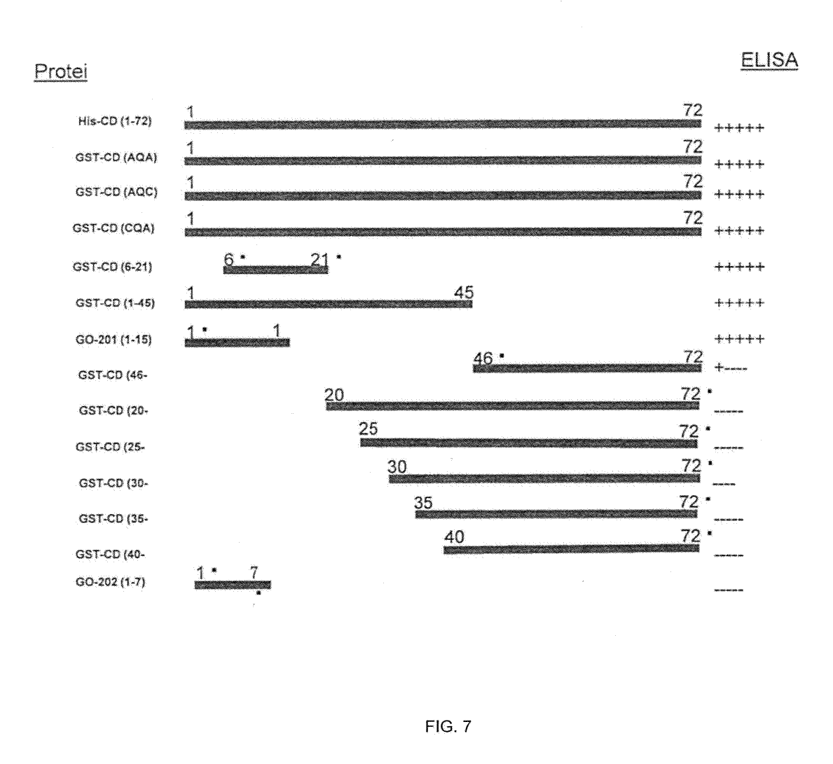

The results of the epitope mapping experiments were summarized and diagrammatically represented in FIG. 7. The summarized results showed that the epitope for the MUC1-CD specific antibody is present between amino acids 8-15 of the MUC1-CD protein. The amino acid sequence of the epitope is YGQLDIFP (SEQ ID NO:12).

Purification of Anti-MUC1-CD MAbs.

Hybridomas were grown in DMEM (Invitrogen) supplemented with 10% FBS containing low bovine IgG. Culture supernatants were passed through protein A-sepharose equilibrated with 50 mM sodium phosphate/300 mM NaCl using an Akta Xpress FPLC system. After washing, antibodies were eluted using 0.1 M citrate buffer, pH 3. Eluted fractions were neutralized, pooled, dialyzed against PBS and concentrated using an Amicon Ultracel 10K filter (Millipore).

Isotyping the Parental Clones of the Hybridoma.

Isotype of the anti-MUC1 antibodies was determined by testing the hybridoma culture supernatants on various anti-immunoglobulin subclass antibodies by sandwich ELISA. Briefly, subclass specific antibodies were coated on to ELISA plates and the test supernatants were subjected to binding with the coated antibodies. The bound antibody was detected by using anti-mouse antibody conjugated to HRP followed by incubation with the HRP substrate. All the three parental clones tested yielded antibody of IgG1k subclass.

### Example 3

### Antibody Sequencing (Examples 1-2)

Total RNA Extraction.

Total RNA was extracted from hybridomas (1E5 cells are sufficient) using a Qiagen kit and quantitated.

First-Round RT-PCR.

QIAGEN® OneStep RT-PCR Kit (Cat No. 210210) was used. RT-PCR was performed with heavy/light chain specific primer sets. For each RNA sample, 12HC+11LC individual RT-PCR reactions were set up using degenerate forward primer mixtures covering the leader sequences of variable regions. Forward primers are used at different concentrations, while reverse primer is at 50 ng per reaction. Note: No restriction sites were engineered into the primers. Reverse primers are located in the constant regions of heavy/light chains. Reaction Setup was:

| | | |
|---|---|---|
| 5x QIAGEN OneStep RT-PCR Buffer: | 5.0 | μl |
| dNTP Mix (containing 10 mM of each dNTP): | 0.8 | μl |
| Primer set: | 0.5 | μl |
| QIAGEN OneStep RT-PCR Enzyme Mix: | 0.8 | μl |
| Template RNA: | 2.0 | μl |
| RNase-free water: | to 20.0 | μl |
| Total volume: | 20.0 | μl |

PCR conditions were as follows:

| | |
|---|---|
| Reverse transcription: | 30 min 50° C. |
| Initial PCR activation step: | 15 min 95° C. |
| Cycling: | 20 cycles of each of |
| | 94° C. for 25 sec |
| | 54° C. for 30 sec |
| | 72° C. for 30 sec |
| Final extension: | 10 min 72° C. |

Second-Round Semi-Nested PCR.

2× PCR mix was used for the second round PCR. The primers are identical to the ones used in the first-round RT-PCR for LC (amount doubled). Seminested reverse primers for HC were used at 100 ng per reaction. Reaction Setup was:

| | |
|---|---|
| 2x PCR mix | 10 μl |
| Primer set | 2 μl |
| First-round PCR product | 8 μl |
| Total volume | 20 μl |

PCR conditions were as follows:

| | |
|---|---|
| Initial denaturing: | 5 min at 95° C., |
| Cycling: | 25 cycles of each of |
| | 95° C. for 25 sec |
| | 57° C. for 30 sec |
| | 68° C. for 30 sec |
| Final extension: | 10 min 68° C. |

After PCR is finished, samples are run onto agarose gel to visualize bands and PCR positive bands are cloned with sequencing of clones.

Summary.

After sequencing more than 15 cloned DNA fragments amplified by nested RT-PCR, several mouse antibody heavy and light chains have been cloned and appear correct. Protein sequence alignment and CDR analysis identifies two distinct heavy chains and one light chain. Their DNA and protein sequences are provided in the attached sequence listing as follows:
SEQ ID NO:1—Heavy chain nucleic acid
SEQ ID NO:2—Heavy chain amino acid
SEQ ID NO:3-5—Heavy Chain CDRs 1-3 chain nucleic acid
SEQ ID NO:6—Light chain nucleic acid
SEQ ID NO:7—Light chain amino acid
SEQ ID NO:8-10—Light chain CDrs 1-3

### Example 4

### Affinity Measurement of Interaction Between Cytoplasmic Domain of MUC1 Protein and Anti-MUC1 Antibodies Using Biacore

The inventors have developed a monoclonal antibody (316.2.4E5.H8.F1, simply called as 4E5) against the cytoplasmic domain of the MUC1 protein that reacts with an epitope adjacent to the CQC motif of the protein. The affinity measurement of interaction between the cytoplasmic domain of MUC1 (MUC1-CD) and 4E5 antibody using surface plasmon resonance (SPR) measurements by BIAcore 3000 was assessed. Binding of a mobile molecule (analyte) to a molecule immobilized on a thin metal film (ligand) changes the refractive index of the film. The angle of extinction of light, reflected after polarized light impinges upon the film, is altered, monitored as a change in detector position for the dip in reflected intensity (SPR). Because the method strictly detects mass, there is no need to label the interacting components, thus eliminating possible changes of their molecular properties. The component that is immobilized to the sensor chip is termed as ‘ligand’ and the component that is injected over the surface is referred to as ‘analyte’.

BIAcore experiments performed involve three major steps such as a) immobilization of the ligand, b) interaction analysis and c) regeneration.

Materials/Reagents.

HBS-EP buffer: 10 mM HEPES, pH 7.4, 150 mM NaCl, 3 mM EDTA, 0.005% v/v P20 (cat. no. BR-1001-88, Biacore). EDC amine coupling reagent: 0.4 M 1-ethyl-3-(3-diethylaminopropyl)carbodiimide hydrochloride in water (part of cat. no. BR-1000-50, Biacore). Ethanolamine solution: 1.0 M ethanolamine-HCl, pH 8.5 (part of cat. no. BR-1000-50, Biacore). NHS amine coupling reagent: 0.1 M N-hydroxysuccinimide in water (part of cat. no. BR-1000-50, Biacore). Sodium acetate buffers for immobilization in the acidic range: 10 mM sodium acetate, pH 5.0 (cat. no. BR-1003-51, Biacore). Glycine-HCl solutions: 10 mM, pH 2.0, Use for regeneration (cat. no. BR-1003-55, Biacore). 50 mM NaOH solution; used for regeneration (cat. no. BR-1003-58, Biacore)

Purified MUC1-CD-His Protein.

Anti-MUC1-CD (CD1, Batch 1) antibody; Anti-MUC1-CD (CD1, batch 5) antibody.

Methods. Ligand Immobilization.

Surface plasmon resonance experiments were performed with a Biacore 3000 instrument. The amine coupling method for immobilization involves activation of carboxyl groups on a dextran-coated chip (CM5 chip) by reaction with 0.2 M N-ethyl-N-(3-diethylamino-propyl)carbodiimide hydrochloride (EDC) and 0.05M N-hydroxysuccinimide (NHS), followed by covalent bonding of the ligands to the chip surface via amide linkages and blockage of excess activated carboxyls with ethanolamine. Reference surfaces were prepared in the same manner, except that the surface was prepared with a non-specific protein such as BSA or MUC1-ECD of the same concentration and the carboxyls are blocked with 1 M ethanolamine. During analysis, each flow cell with the ligand is paired with an adjacent flow cell on the chip as a reference.

The final concentration of bound ligand is expressed in response (resonance) units (RU). For a typical kinetic analysis, the amount of ligand to be immobilized should yield a binding of 50-150 RU of analyte and it is determined using the following theoretical formula. The amount of ligand to immobilize for any application depends on the relative molecular weights of the ligand and analyte and on the sensitivity of the Biacore system. Since the SPR response is directly proportional to the mass and concentration of material at the surface, the theoretical analyte binding capacity of a given surface is related to the amount of ligand immobilized and as follows:

$$analyte binding capacity ( R U ) = \frac{analyte MW}{ligand MW} \times immobilized ligand level ( R U ) \times Sm$$

where Sm refers to the molar stoichiometry or valency of the ligand. For example, if the ligand molecular weight is 100,000 daltons and the analyte molecular weight is 50,000 daltons, immobilizing 5,000 RU of ligand will give a theoretical analyte binding capacity of 2,500 RU assuming that the ligand is 100% active and the binding is with 1:1 stoichiometric ratio. In this case, the ligand is the antibody with a MW of 150,000 and the analyte is His-CD with a molecular weight of 8,000. Accordingly, to obtain a response of 150 RU, the antibody should be immobilized to 937 RU.

If His-CD with MW of 8000 is used as the ligand and the antibody as the analyte, it requires only 16 RU of the ligand to be immobilized. However, it is very low which might lead to a risk of not yielding any binding. Therefore, the ligand was immobilized to 150 RU arbitrarily and tested in the initial binding.

Ideally, for efficient immobilization, a pH scouting experiment needs to be performed with ligand in buffers of different pH. However, our study is aimed at analyzing the kinetics of binding, it requires only 150 RU of ligand to be coupled and therefore, immobilization was performed in only one pH (i.e., acetate buffer, pH 5.0)

Immobilization of Anti MUC1-CD was performed in flow cell 2 as mentioned above by injecting 20 μg/ml of antibody in acetate buffer, pH 5.0 through the flow cell followed by blocking the reactive ester with ethanolamine. As a negative control, flow cell 1 was used as reference where MUC1-ECD antibody (8E1) was coupled using 20 μg/ml of the antibody.

In a different set of experiment, 10 μg/ml of His-CD was coupled with the same conditions in flow cell 2 (FC2) and BSA in FC1 as reference. Since His-CD is known to dimerize and even oilgomerize at room temperature, before coupling it was resuspended in Acetate buffer, pH 5.0 containing 2 mM DTT. After coupling the ligand, 0.5% BSA in HBS-EP buffer was injected through FC1 and FC2 to reduce non-specific binding and washed with the same buffer 5 minutes.

Testing the Binding Surface and Initial Binding.

The immobilized sensor chip surface binding capacity was tested by injecting 3 concentrations of the analyte that covers at least 10-fold difference between the highest and the lowest concentrations tested. The analyte in HBS-EP buffer, pH 7.4 was injected at a flow rate of 5 μl/min for 5 minutes and washed with the same buffer for 5 minutes at a rate of 5 μl/min for 5 minutes and regenerated using 10 mM glycine-HCl, pH 2.0 for 5 minutes before testing different concentration of the analyte. The real time binding could be seen from the sensogram. This process enables the testing of the binding surface and the data obtained is used for determining the lowest and the highest concentration to be used in the kinetic analysis.

Three different experiments were performed. In the first experiment, anti-MUC1-CD antibody, (CD1, preparation batch 1) was coupled on the CM5 chip and His-CD (as analyte) was injected at 50, 200 and 1000 nM in HBS-EP buffer, pH 7.4.

In the second experiment, His-CD was coupled to the chip and anti-MUC1-CD was injected at 10, 50 and 100 nM in HBS-EP buffer, pH 7.4. The third experiment was repeated with the same conditions using an anti-MUC1-CD antibody (CD1, preparation batch 5).

Results.

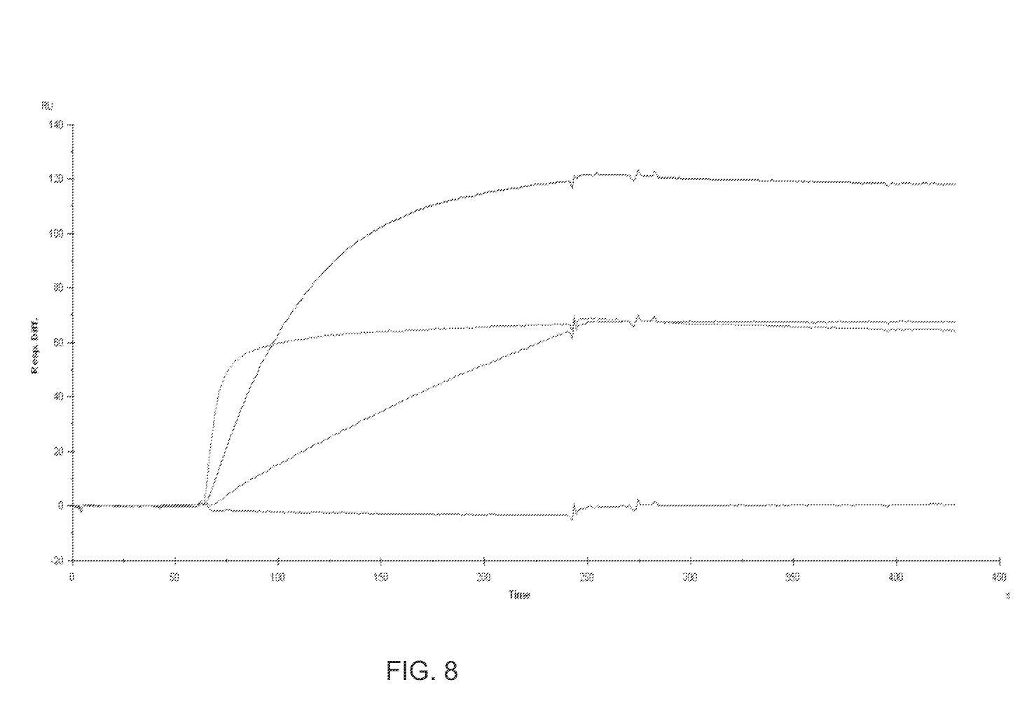

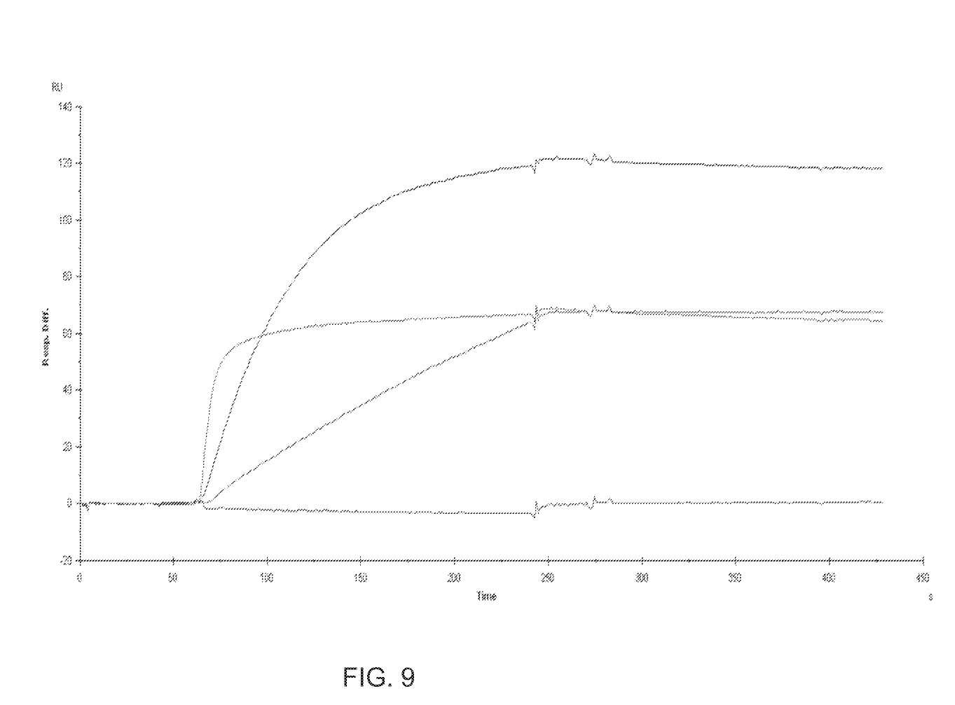

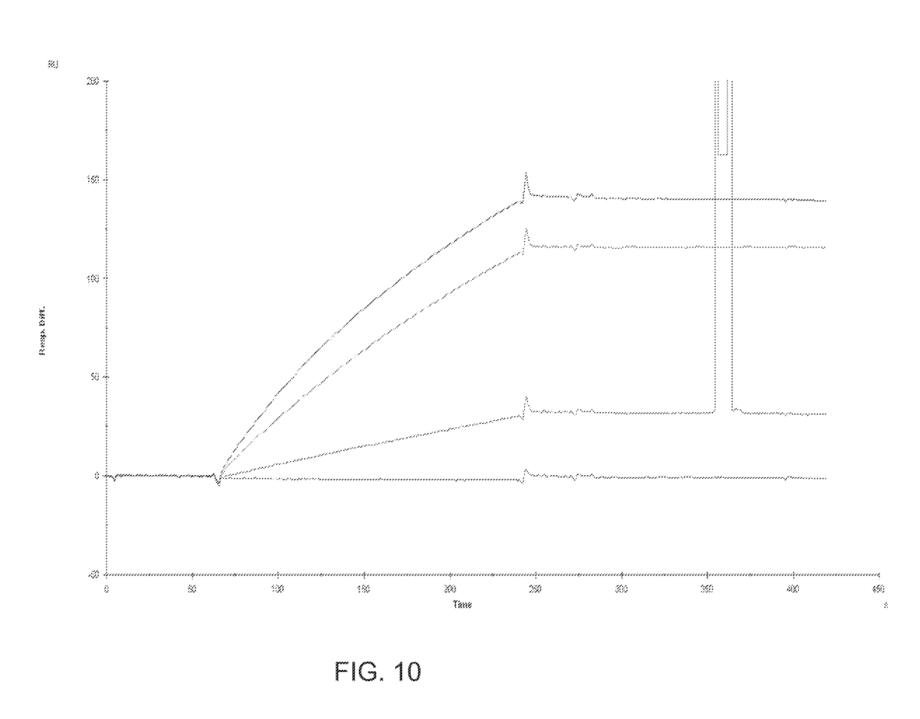

The real time binding as it happened has been shown in the following sensograms (FIGS. 8-10). As shown in FIG. 8, His-CD showed an expected binding of 130 RU at a maximum concentration of 200 nM where as increasing the concentration up to 1000 nM did not show any improved binding rather inhibited the binding. Therefore, for kinetic studies 7 concentrations were chosen between 10 nM and 200 nM. Similarly, as seen in FIGS. 9-10, the expected 150 RU was achieved with the maximum concentration of 100 nM of anti-MUC1-CD antibody. Therefore, for kinetic study the concentrations chosen were between 5 nM and 100 nM.

Kinetic Analysis of His-CD and Anti-MUC1-CD Interaction.

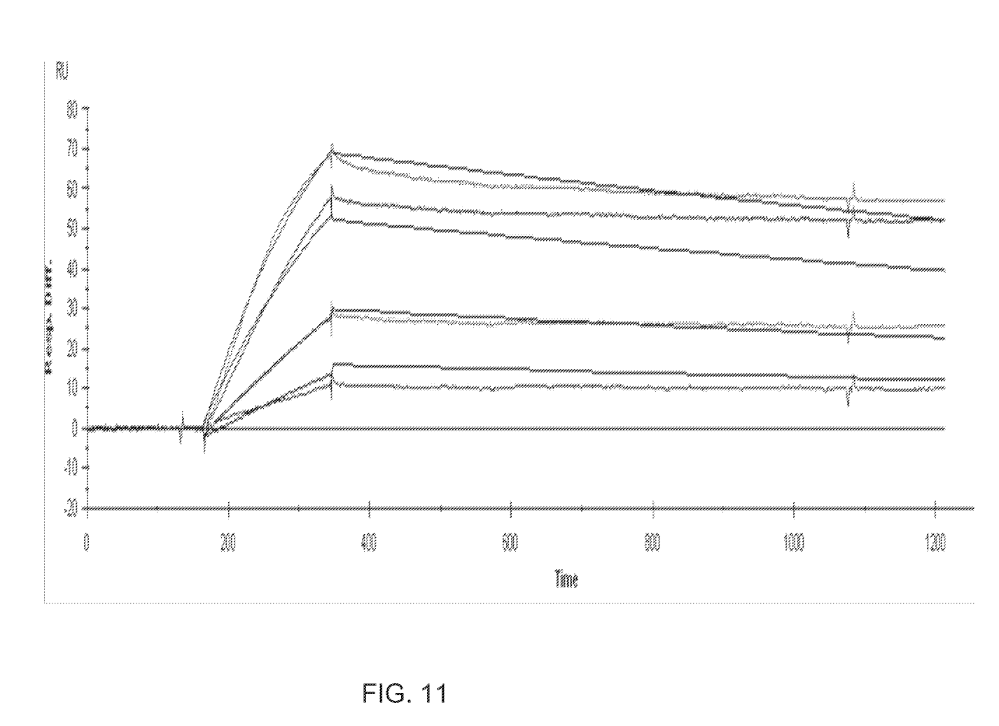

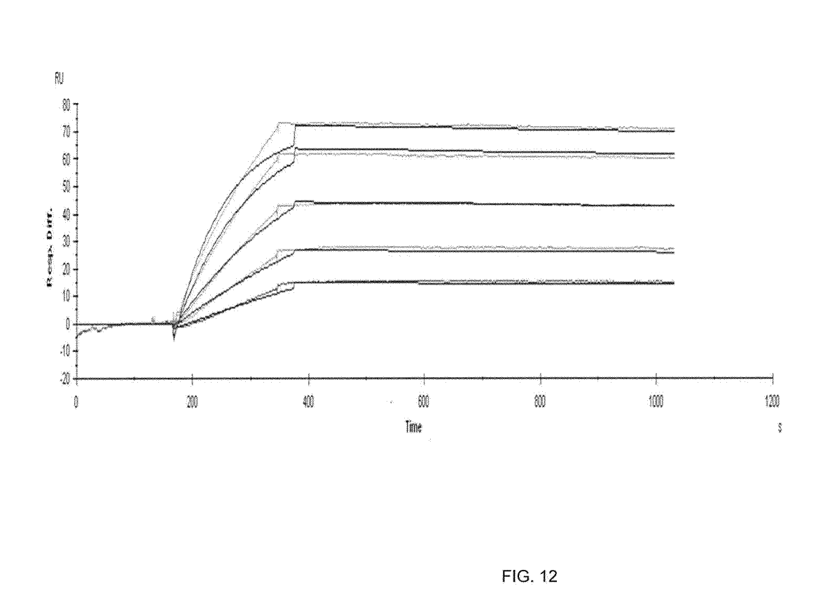

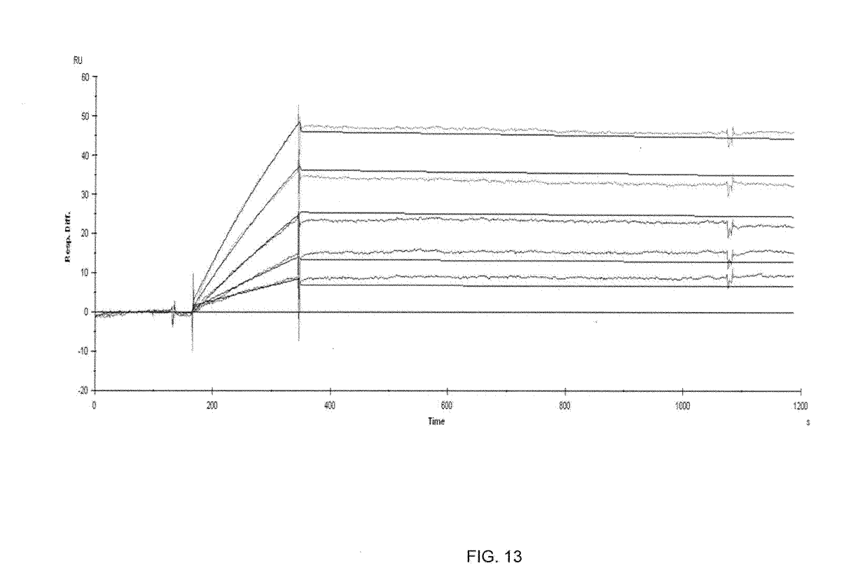

Kinetic analyses were performed by immobilizing His-CD on the sensor chip and injecting the MUC1-CD antibody, or the antibody was immobilized and Hi-CD was passed through the chip. The analyte was passed through the chip at 30 μl/min for 3 minutes with 15 minutes dissociation time. The chip was regenerated between each binding with glycine-HCl, pH 2.0 for 30 seconds at a rate of 30 gl/min. Based on the initial binding analysis, the concentrations of analyte were chosen as mentioned above. The data obtained from the sensograms were analyzed using BIAevaluation software. The data were analyzed using global fitting procedures applying the 1:1 Langmuir binding model. In the analysis, kₐ and k_{d} were globally fitted and the equilibrium constant (K_{A}) was obtained from the ratio of the rate constants (K_{A}=kₐ/k_{d}). The results of kinetic analyses have been shown in FIGS. 11-13 as sensogram and the analyzed results have presented in Tables 12-14.

TABLE 12
Data from kinetics of His-CD to immobilized Anti MUC1-CD (batch 1)

| Anti-CD | | | Rmax | RI | Conc of | KA | | Req | | |
|---|---|---|---|---|---|---|---|---|---|---|
| concn | ka (1/Ms) | kd (1/s) | (RU) | (RU) | analyte | (1/M) | KD (M) | (RU) | kobs (1/s) | Chi² |
| | 8.01E+04 | 3.21E−04 | 122 | | | 2.49E+08 | 4.01E−09 | | | 9.42 |
| Anti-CD | | | | 0 | 0 | | | 0 | 3.21E−04 | |
| (0 nM) | | | | | | | | | | |
| Anti-CD | | | | −2.28 | 1.00E−08 | | | 86.7 | 1.12E−03 | |
| (10 nM) | | | | | | | | | | |
| Anti-CD | | | | −1.8 | 2.00E−08 | | | 101 | 1.92E−03 | |
| (20 nM) | | | | | | | | | | |
| Anti-CD | | | | 1.65 | 4.00E−08 | | | 110 | 3.52E−03 | |
| (40 nM) | | | | | | | | | | |
| Anti-CD | | | | 0.748 | 6.00E−08 | | | 114 | 5.12E−03 | |
| (60 nM) | | | | | | | | | | |

The binding affinity (KD) in this run was calculated to be 4.01 nM, as highlighted in yellow in the table.

TABLE 13
Data from kinetics of Anti MUC1-CD (batch 1) to immobilized His-CD

| | | | Rmax | | Conc of | | | | kobs | |
|---|---|---|---|---|---|---|---|---|---|---|
| | ka (1/Ms) | kd (1/s) | (RU) | RI (RU) | analyte | KA (1/M) | KD (M) | Req (RU) | (1/s) | Chi² |
| | 2.00E+05 | 5.00E−05 | 79 | | | 4.00E+09 | 2.50E−10 | | | 1.61 |
| His-CD 5 nM | | | | −1.89 | 5.00E−09 | | | 75.2 | 1.05E−03 | |
| His-CD 10 nM | | | | −1.14 | 1.00E−08 | | | 77.1 | 2.05E−03 | |
| His-CD 20 nM | | | | −1.98 | 2.00E−08 | | | 78 | 4.05E−03 | |
| His-CD 40 nM | | | | −5.04 | 4.00E−08 | | | 78.5 | 8.06E−03 | |
| His-CD 60 nM | | | | −7.32 | 6.00E−08 | | | 78.7 | 0.0121 | |

TABLE 14
Data from kinetics of Anti MUC1-CD (batch 5) to immobilized His-CD

| | | | Rmax | RI | Conc of | | | Req | | |
|---|---|---|---|---|---|---|---|---|---|---|
| | ka (1/Ms) | kd (1/s) | (RU) | (RU) | analyte | KA (1/M) | KD (M) | (RU) | kobs (1/s) | Chi² |
| | 2.81E+04 | 4.37E−05 | 138 | | | 6.43E+08 | 1.55E−09 | | | 2.15 |
| His-CD | | | | 0 | 0 | | | 0 | 4.37E−05 | |
| 0 nM | | | | | | | | | | |
| His-CD | | | | 2.78 | 80 nM | | | 136 | 2.29E−03 | |
| 80 nM | | | | | | | | | | |
| His-CD | | | | 1.35 | 60 nM | | | 135 | 1.73E−03 | |
| 60 nM | | | | | | | | | | |
| His-CD | | | | −0.194 | 40 nM | | | 133 | 1.17E−03 | |
| 40 nM | | | | | | | | | | |
| His-CD | | | | 0.907 | 20 nM | | | 128 | 6.06E−04 | |
| 20 nM | | | | | | | | | | |
| His-CD | | | | 1.66 | 10 nM | | | 120 | 3.25E−04 | |
| 10 nM | | | | | | | | | | |

The results obtained from three different experiments yielded affinity of interaction (K_{D} value) of 4.01×10⁻⁹, 0.25×10⁻⁹ and 1.5×10⁻⁹ M respectively (4.01 nM, 0.25 nM and 1.5 nM). These differences could be attributed to the experimental variations such as inclusion of DTT to maintain the monomeric state of His-CD. When the antibody was immobilized on the surface of the sensor chip and MUC1-His-CD was injected through the flow cell and DTT could be used to monomerize the analyte as it might destroy the antibody coupled on the surface. However, the affinity of interaction obtained remained in nanomolar level (i.e., 0.25-4.01 nM).

Conclusions.

A monoclonal antibody against the cytoplasmic domain of MUC1-C (MUC1-CD; CD1) has generated and the reactivity of the antibody against the antigen has been verified by ELISA and it was further characterized. The experiments presented in this report were aimed at determining the affinity of interaction of the antibody against its antigen using BIACORE analysis. The affinity measurements were performed using surface plasmon resonance with BIAcore 3000. The affinity of interaction (K_{D}) obtained from this study shows a very high affinity, in the nanomolar range (0.25-4.01 nM).

### Example 5

### Antibody Sequencing and IHC for CD-2

Methods. Total RNA Extraction.

Total RNA was extracted from hybridomas using Qiagen kit.

First-Round RT-PCR.

QIAGEN® OneStep RT-PCR Kit (Cat No. 210210) was used. RT-PCR was performed with primer sets specific for the heavy and light chains. For each RNA sample, 12 individual heavy chain and 11 light chain RT-PCR reactions were set up using degenerate forward primer mixtures covering the leader sequences of variable regions. Reverse primers are located in the constant regions of heavy and light chains. No restriction sites were engineered into the primers. Reaction setup was:

| | |
|---|---|
| 5x QIAGEN ® OneStep RT-PCR Buffer | 5.0 μl |
| dNTP Mix (containing 10 mM of each dNTP) | 0.8 μl |
| Primer set | 0.5 μl |
| QIAGEN ® OneStep RT-PCR Enzyme Mix | 0.8 μl |
| Template RNA 2.0 ul; RNase-free water to | 20.0 μl |
| Total volume | 20.0 μl |

PCR conditions were:

Reverse transcription: 50° C., 30 min

Initial PCR activation: 95° C., 15 min

20 cycles of:
94° C., 25 sec
54° C., 30 sec
72° C., 30 sec
Final extension: 72° C., 10 min

Second-Round Semi-Nested PCR.

The RT-PCR products from the first-round reactions were further amplified in the second-round PCR. 12 individual heavy chain and 11 light chain RT-PCR reactions were set up using semi-nested primer sets specific for antibody variable regions. Reaction setup was:

| | |
|---|---|
| 2x PCR mix | 10 μl |
| Primer set | 2 μl |
| First-round PCR product | 8 μl |
| Total volume | 20 μl |

PCR conditions were:

Initial denaturing of 5 min at 95° C.,

25 cycles of:
95° C. for 25 sec,
57° C. for 30 sec,
68° C. for 30 sec.

Final extension is 10 min 68° C.

After PCR is finished, PCR reaction samples are run onto agarose. TOPO cloned PCR positive bands are the PCR-amplified, followed by gel electrophoresis and recovered from agarose gel. Twenty-four clones were sequenced and CDR analysis was performed.

Immunohistochemistry.

Slides were incubated in a 60° C. oven to melt the paraffin for 15 minutes and then transferred to xylene substitute for 10 minutes and deparaffinized (2 rounds of xylene, 100% ethanol, 95% ethanol, 75% ethanol, 50% ethanol, 25% ethanol and distilled water). Antigen retrieval (HIER: Heat induced epitope retrieval) was performed using a Biocare Decloaker (pressure cooker). The slide racks were placed in Diva Decloaker solution (Biocare) pH 6.0, incubate at 100° C. for 35 minutes and then cool to 85° C. for 10 minutes. Slides were transferred to DI water and stained using reagents from Biocare Medical using the Intellipath Auto Stainer (automatic stainer):
Block: with Peroxidase 1 for 10 min
Washed with TBS+tween
Block: background sniper
Washed with TBS+tween
ab: CD2 1:100 for 2 hrs
Washed with TBS+tween
Secondary: mouse secondary reagent (15 min)
Washed with TBS+tween
Secondary: universal HRP Reagent (15 min)
Washed with TBS+tween
Chromagen (mixed online): DAB (5 min)
Washed with DI Water×2
Counterstain: hematoxylin (3 min)
Washed with TBS+tween
   Slides are then dehydrated, mounted and evaluated.

Results.

After sequencing, several mouse antibody heavy and light chains have been identified. Antibody CDR analysis identifies one heavy chain and one light chain. The variable heavy chain is illustrated below:

----CDR1---> <--CDR2--> <---CDR3----
(SEQ ID NOS: 17-19)
GFNIEDHY...._IDPEDGQT.._ARNYPYALDY
Amino Acid Sequence
(SEQ ID NO: 14)
EVQLQQSGAELVKPGASVKLSCTASGFNIEDHYMHWVKQRTEQGLDWIGRIDPEDGQTKYD
PKFQGKATITVDTSSNTAYLQLSSLTSEDTAVYYCARNYPYALDYWGQGTSVTVSS
Nucleotide Sequence
(SEQ ID NO: 13)
GAGGTTCAGCTGCAGCAGTCTGGGGCAGAGCTTGTGAAGCCAGGGGCCTCAGTCAAGTT
GTCCTGCACAGCTTCCGGCTTCAACATTGAAGACCACTATATGCATTGGGTGAAGCAGAG
GACTGAACAGGGCCTGGACTGGATTGGAAGGATTGATCCTGAGGATGGACAAACTAAAT
ATGACCCGAAATTCCAGGGCAAGGCCACTATAACTGTCGACACGTCCTCCAACACAGCCT
ACCTGCAGCTCAGCAGCCTGACATCTGAGGACACTGCCGTCTATTATTGTGCTAGAAACT
ATCCCTATGCTTTGGACTATTGGGGACAAGGAACCTCAGTCACCGTCTCCTCA

The variable light chain is shown below:

----CDR1---> <--CDR2--> <---CDR3----
(SEQ ID NOS: 20-21)
QSLLYSSDQKNY_WAS......._QQYYRYPFT
Amino Acid Sequence
(SEQ ID NO: 16)
DIVMSQSPSSLAVSVGEKVTLSCKSSQSLLYSSDQKNYLAWYQQKPGQSPKLLIYWASTRES
GVPDRFTGSGSGTDFTLTISSVKAEDLAVYYCQQYYRYPFTFGSGTQLEIK
Nucleotide Sequence
(SEQ ID NO: 15)
GACATTGTGATGTCACAGTCTCCATCCTCCCTAGCTGTGTCAGTTGGAGAGAAGGTTACT
CTGAGCTGCAAGTCCAGTCAGAGCCTTTTATATAGTAGCGACCAAAAGAACTACTTGGCC
TGGTACCAGCAGAAACCAGGGCAGTCTCCTAAACTGCTGATTTACTGGGCATCCACTAGG
GAATCTGGGGTCCCTGATCGCTTCACAGGCAGTGGATCTGGGACAGATTTCACTCTCACC
ATCAGCAGTGTGAAGGCTGAAGACCTGGCAGTTTATTACTGTCAGCAATATTATAGGTAT
CCATTCACGTTCGGCTCGGGGACACAGTTGGAAATAAAAC

Immunohistochemistry.

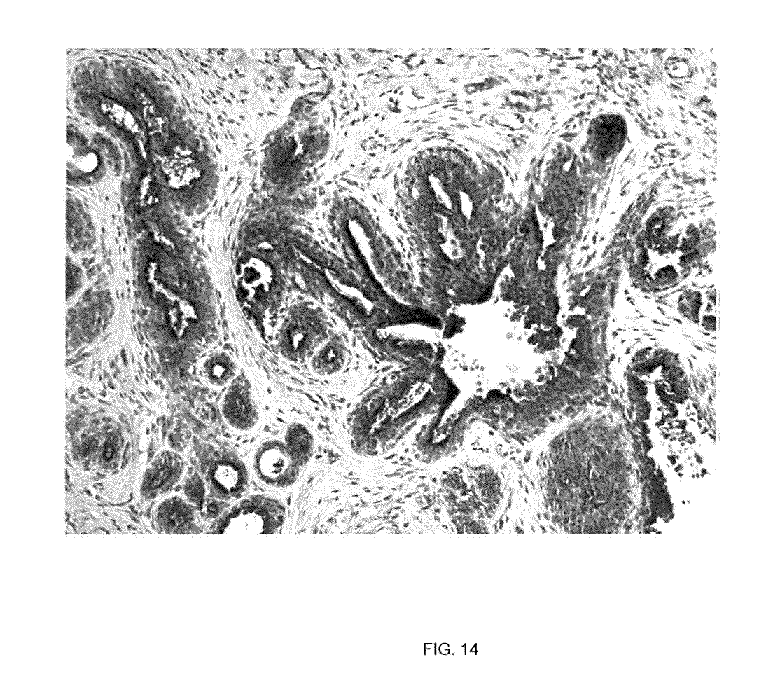

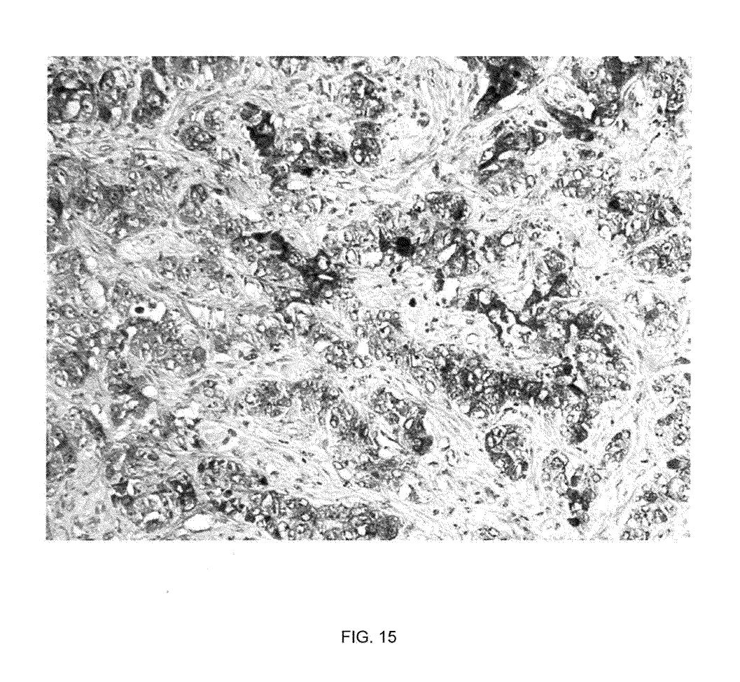

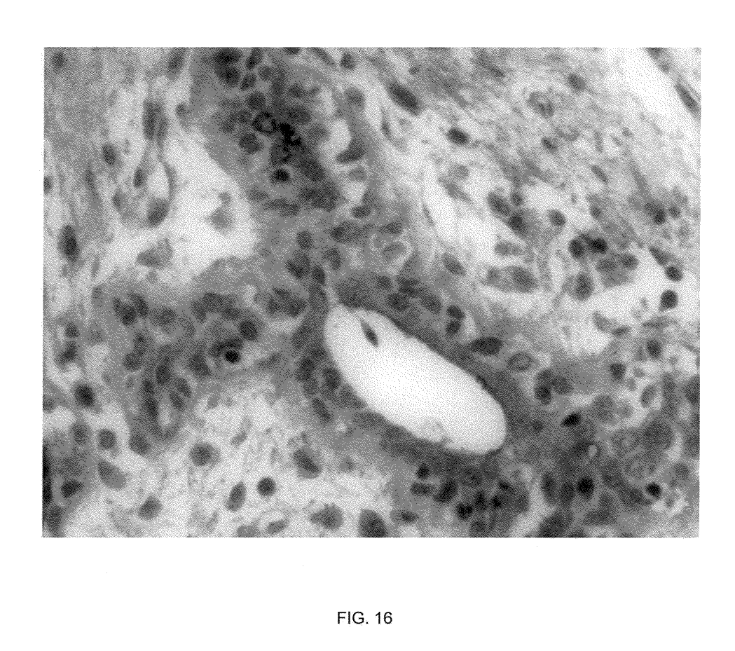

To determine whether anti-MUC1-CD (CD2) antibody can be used for detecting MUC1-C expression in fixed human tumor tissues, the inventors analyzed formalin-fixed sections of human breast carcinoma. Staining of human breast carcinoma tissues with anti-MUC1-CD (CD2) antibody was clearly evident and in some patients is very strong staining. Staining was also strong to inner linings of the duct (columnar epithelial cells). Staining was restricted to epithelial cells and not the stromal components supporting the specificity of reactivity with MUC1-C expressing cells. See FIGS. 14-16.)

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
“Antibodies: A Laboratory Manual,” Cold Spring Harbor Press, Cold Spring Harbor, N.Y., 1988.
Abbondanzo et al., Am. J. Pediatr. Hematol. Oncol., 12(4), 480-489, 1990.
Abe and Kufe, Cancer Res., 49(11):2834-2839, 1989.
Ahmad et al., Nat. Cell Biol., 9:1419-1427, 2007.
Allred et al., Arch. Surg., 125(1), 107-113, 1990.
Atherton et al., Biol. of Reproduction, 32, 155-171, 1985.
Baldus et al., Clin. Cancer Res., 10(8):2790-2796, 2004.
Beidler et al., J. Immunol., 141(11):4053-4060, 1988.
Brown et al., J. Immunol. Meth., 12; 130(1), 111-121, 1990.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm., 74(2):425-433, 1977.
De Jager et al., Semin. Nucl. Med. 23(2), 165-179, 1993.
Dholakia et al., J. Biol. Chem., 264, 20638-20642, 1989.
Doolittle and Ben-Zeev, Methods Mol. Biol., 109, 215-237, 1999.
Duraisamy et al., Gene, 373:28-34, 2006.
EP Application 125,023
EP Application 171,496
EP Application 173,494
EP Application 184,187
Gefter et al., Somatic Cell Genet., 3:231-236, 1977.
Gefter et al., Somatic Cell Genet., 3:231-236, 1977.
Goding, In: Monoclonal Antibodies: Principles and Practice, 2d ed., Orlando, Fla., Academic Press, 60-61, 65-66, 71-74, 1986.
Gulbis and Galand, Hum. Pathol. 24(12), 1271-1285, 1993.
Guo et al., Sci. Transl. Med. 3:99 ra85, 2001.
Hodel et al., Mol. Cell, 10(2):347-58, 2002.
Huang et al., Cancer Biol Ther., 2:702-706, 2003.
Huang et al., Cancer Res., 65:10413-10422, 2005.
Jones et al., Nature, 321:522-525, 1986.
Kau et al., Nat. Rev. Cancer, 4(2):106-17, 2004.
Khatoon et al., Ann. of Neurology, 26, 210-219, 1989.
King et al., J. Biol. Chem., 269, 10210-10218, 1989.
Kinlough et al., J. Biol. Chem., 279(51):53071-53077, 2004.
Kinoshita et al., Biochem. Biophys. Res. Commun., 394:205-210, 2010.
Kohler and Milstein, Eur. J. Immunol., 6, 511-519, 1976.
Kohler and Milstein, Nature, 256, 495-497, 1975.
Kufe et al., Hybridoma, 3:223-232, 1984.
Kyte and Doolittle, J. Mol. Biol., 157(1):105-132, 1982.
Leng et al., J. Biol. Chem., 282:19321-19330, 2007.
Levitan et al., J. Biol. Chem., 280:33374-33386, 2005.
Li et al., Cancer Biol. Ther., 2:187-193, 2003b.
Li et al., J. Biol. Chem., 276:35239-35242, 2001.
Li et al., J. Biol. Chem., 276:6061-6064, 2001.
Li et al., Mol. Cancer. Res., 1:765-775, 2003c.
Li et al., Mol. Cell. Biol., 18:7216-7224, 1998.
Li et al., Oncogene, 22:6107-6110, 2003a.
Ligtenberg et al., J. Biol. Chem., 267, 6171-6177, 1992.
Macao, Nat. Struct. Mol. Biol., 13, 71-76, 2006.
Merlo et al., Cancer Res., 49, 6966-6971, 1989.
Morrison, Science, 229(4719):1202-1207, 1985.
Nakamura et al., In: Enzyme Immunoassays: Heterogeneous and Homogeneous Systems, Chapter 27, 1987.
O'Shannessy et al., J. Immun. Meth., 99, 153-161, 1987.
Owens and Haley, J. Biol. Chem., 259, 14843-14848, 1987.
Owens and Haley, J. Biol. Chem., 259:14843-14848, 1987.
PCT Application PCT/US86/02269
PCT Application WO 86/01533
Percipalle et al., J. Mol. Biol., (4):722-32, 1997.
Perey et al., Cancer Res., 52(22):6365-6370, 1992.
Persic et al., Gene 187:1, 1997
Posner et al., Hybridoma 6, 611-625, 1987.
Potter and Haley, Meth. Enzymol., 91, 613-633, 1983.
Raina et al., EMBO J., 25:3774-3783, 2006.
Raina et al., J. Biol. Chem., 279:20607-20612, 2004.
Remington's Pharmaceutical Sciences, 15^{th} Ed., 3:624-652, 1990.
Ren et al., Cancer Cell, 5:163-175, 2004.
Ren et al., J. Biol. Chem., 277:17616-17622, 2002.
Ryan and Wente, Curr. Opin. Cell Biol., 12(3):361-71, 2000.
Schroeder et al., J. Biol. Chem., 276(16):13057-13064, 2001.
Schroeder et al., Oncogene, 23:5739-5747, 2004.
Shaw et al., J. Natl. Cancer Inst., 80(19):1553-1559, 1988.
Siddiqui et al., Proc. Natl. Acad. Sci. USA, 85:2320-2323, 1988.
Suh and Gumbiner, Exp. Cell Res., 290(2):447-56, 2003.
Sun et al., J. Steroid Biochem., 26(1):83-92, 1987.
Tang et al., J. Biol. Chem., 271:28324-28330, 1996.
Truscott et al., J Cell Biol., 163(4):707-713, 2003.
Verhoeyen et al., Science, 239(4847):1534-1536, 1988.
Vermeer et al., Nature, 422(6929):322-6, 2003.
Wawrzynczak & Thorpe, In: Immunoconjugates, Antibody Conjugates In Radioimaging And Therapy Of Cancer, Vogel (Ed.), NY, Oxford University Press, 28, 1987.
Wei et al., Cancer Cell, 7:167-178, 2005.
Weis, Cell, 112(4):441-51, 2003.
Wen et al., J. Biol. Chem., 278:38029-38039, 2003.
Wood et al., J. Clin. Lab. Immunol., 17(4):167-171, 1985.
Yamamoto et al., J. Biol. Chem., 272:12492-12494, 1997.
Yin et al., J. Biol. Chem., 278:35458-35464, 2003.
Yin et al., J. Biol. Chem., 279:45721-45727, 2004.
Yin et al., J. Biol. Chem., 282:257-266, 2007.
Young et al., Cell. 112(1):41-50, 2003.

1. A method of detecting MUC1 in a cell or tissue comprising (a) contacting said cell or tissue with an antibody reagent that binds immunologically to the cytoplasmic domain of MUC1; and (b) detecting said antibody reagent bound to said cell or tissue.

2. The method of claim 1, wherein said cell or tissue is a cancer cell or tissue.

3. (canceled)

4. The method of claim 1, wherein said cell or tissue is from a tumor biopsy from a subject.

5. (canceled)

6. The method of claim 5, wherein tumor biopsy is from a patient not having been previously diagnosed with cancer.

7. The method of claim 5, wherein tumor biopsy is from a patient not having been previously diagnosed with a MUC1-positive cancer.

8. The method of claim 5, wherein tumor biopsy is from a patient been previously diagnosed with cancer.

9. The method of claim 5, wherein tumor biopsy is from a patient having been previously diagnosed with a MUC1-positive cancer.

10. The method of claim 5, wherein tumor biopsy is from a patient undergoing or having previously undergone cancer treatment.

11. The method of claim 5, further comprising contacting a second cell or tissue from a second tumor biopsy with said reagent.

12. The method of claim 1, wherein said reagent comprises a recombinant antibody, a single chain antibody or an antibody fragment.

13-18. (canceled)

19. The method of claim 1, wherein said reagent is labeled with a detectable moiety.

20. (canceled)

21. The method of claim 1, wherein said reagent is not labeled, and detecting is accomplished using a secondary binding agent that is labeled with a detectable moeity, the secondary binding agent having affinity for said antibody reagent.

22. The method of claim 1, wherein detection comprises a ELISA or RIA, a competitive assay or immunohistochemistry.

23-26. (canceled)

27. The method of claim 1, wherein said reagent is an antibody or antibody fragment comprising a light chain variable sequence according to SEQ ID NO:4 or a sequence having 80% identity to SEQ ID NO:4, and a heavy chain sequence according to SEQ ID NO:2 or a sequence having 80% identity to SEQ ID NO:2.

28. The method of claim 27, wherein said antibody or antibody fragment is encoded by a light chain variable sequence according to SEQ ID NO:3 or a sequence having 70% identity to SEQ ID NO:3, and a heavy variable chain sequence according to SEQ ID NO:1 or a sequence having 70% identity to SEQ ID NO:1.

29. The method of claim 1, wherein said reagent is an antibody or antibody fragment comprising light chain variable CDR sequences according to SEQ ID NO:8-10.

30. The method of claim 1, wherein said reagent is an antibody or antibody fragment comprising heavy chain variable CDRs sequences according to SEQ ID NO:3-5.

31. A monoclonal antibody comprising a light chain variable sequence according to SEQ ID NO:4 or a sequence having 80% identity to SEQ ID NO:4, and a heavy chain sequence according to SEQ ID NO:2 or a sequence having 80% identity to SEQ ID NO:2.

32. The antibody of claim 31, wherein said antibody or antibody fragment is encoded by a light chain variable sequence according to SEQ ID NO:3 or a sequence having 70% identity to SEQ ID NO:3, and a heavy variable chain sequence according to SEQ ID NO:1 or a sequence having 70% identity to SEQ ID NO:1.

33. A monoclonal antibody comprising light chain variable CDR sequences according to SEQ ID NO:8-10.

34. A monoclonal antibody comprising heavy chain variable CDRs sequences according to SEQ ID NO:3-5.

35. A method of treating cancer comprising contacting a MUC1-positive cancer cell in a subject with that binds immunologically to the cytoplasmic domain of MUC1.

36. The method of claim 35, wherein said MUC1-positive cancer cell is a solid tumor cell.

37. The method of claim 36, wherein said solid tumor cell is a lung cancer cell, brain cancer cell, head & neck cancer cell, breast cancer cell, skin cancer cell, liver cancer cell, pancreatic cancer cell, stomach cancer cell, colon cancer cell, rectal cancer cell, uterine cancer cell, cervical cancer cell, ovarian cancer cell, testicular cancer cell, skin cancer cell, or esophageal cancer cell.

38. The method of claim 35, wherein said MUC1-positive cancer cell is a leukemia or myeloma.

39. The method of claim 38, wherein said leukemia or myeloma is acute myeloid leukemia, chronic myelogenous leukemia or multiple myeloma.

40. The method of claim 35, further comprising contacting said MUC1-positive cancer cell with a second anti-cancer agent or treatment.

41. (canceled)

42. The method of claim 40, wherein said second anti-cancer agent inhibits an intracellular MUC1 function.

43-45. (canceled)

46. The method of claim 35, wherein said antibody is a single chain antibody, a single domain antibody, a chimeric antibody or a Fab fragment.

49. (canceled)

50. The method of claim 35, wherein said antibody is a recombinant antibody having specificity for the MUC1 ECD and a distinct cancer cell surface antigen.

51. The method of claim 35, wherein said antibody is a murine antibody, murine IgG antibody, a humanized antibody, or a humanized IgG antibody.

52-54. (canceled)

55. The method of claim 35, wherein said antibody further comprises an antitumor drug linked thereto.

56. The method of claim 55, wherein said antitumor drug is linked to said antibody through a photolabile linker or an enzymatically-cleaved linker.

57-58. (canceled)

59. The method of claim 35, wherein said antibody further comprises a label.

60. (canceled)

61. The method of claim 35, wherein said antibody is conjugated to a liposome or a nanoparticle.


SEQUENCE LISTING (21 sequences)

SEQ ID NO: 1 - DNA, 348 bp, Mus musculus
gaggttcagctgcagcagtctggggcagaacttgtgaggcctggggcctcagtcaagttgtcctgcacagcttctggcctcaacattcaagactactttatacactgggtgaagcagaggcctgaacagggcctggagtggattggaaggattgatcctgaaaatggtgatactgaatatgccccgaagttccagggcaaggccactatgactgcagacacatcctccaacacagcctacctgcagctcagcagcctgacatctgaggacactgccgtctattactgtactagcggtagtagatttcgggactactggggtcagggaacctcagtcaccgtctcctca

SEQ ID NO: 2 - protein, 116 aa, Mus musculus
EVQLQQSGAELVRPGASVKLSCTASGLNIQDYFIHWVKQRPEQGLEWIGRIDPENGDTEYAPKFQGKATMTADTSSNTAYLQLSSLTSEDTAVYYCTSGSRFRDYWGQGTSVTVSS

SEQ ID NO: 3 - protein, 8 aa, Mus musculus
GLNIQDYF

SEQ ID NO: 4 - protein, 8 aa, Mus musculus
IDPENGDT

SEQ ID NO: 5 - protein, 9 aa, Mus musculus
TSGSRFRDY

SEQ ID NO: 6 - DNA, 270 bp, Mus musculus
gagtcagtttctcctgcagggccagtcagagcattggcacaagcattcactggtatcagcaaagaacaaatggttctccaaggcttctcataaagtttgcttctgagtctatctctgggatcccttccaggtttagtggcagtggatcagggacagattttactcttagcatcaacagtgtggagtctgaagatattgcagattattactgtcaacaaagtaatagctggccaatcacgttcggtgctgggaccaggctggagctgaaac

SEQ ID NO: 7 - protein, 89 aa, Mus musculus
VSFSCRASQSIGTSIHWYQQRTNGSPRLLIKFASESISGIPSRFSGSGSGTDFTLSINSVESEDIADYYCQQSNSWPITFGAGTRLELK

SEQ ID NO: 8 - protein, 6 aa, Mus musculus
QSIGTS

SEQ ID NO: 9 - protein, 3 aa, Mus musculus
FAS

SEQ ID NO: 10 - protein, 9 aa, Mus musculus
QQSVSWPIT

SEQ ID NO: 11 - protein, 159 aa, Homo sapiens
GSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 12 - protein, 8 aa, Artificial Sequence
  Synthetic peptide
YGQLDIFP

SEQ ID NO: 13 - DNA, 351 bp, Mus musculus
gaggttcagctgcagcagtctggggcagagcttgtgaagccaggggcctcagtcaagttgtcctgcacagcttccggcttcaacattgaagaccactatatgcattgggtgaagcagaggactgaacagggcctggactggattggaaggattgatcctgaggatggacaaactaaatatgacccgaaattccagggcaaggccactataactgtcgacacgtcctccaacacagcctacctgcagctcagcagcctgacatctgaggacactgccgtctattattgtgctagaaactatccctatgctttggactattggggacaaggaacctcagtcaccgtctcctca

SEQ ID NO: 14 - protein, 117 aa, Mus musculus
EVQLQQSGAELVKPGASVKLSCTASGFNIEDHYMHWVKQRTEQGLDWIGRIDPEDGQTKYDPKFQGKATITVDTSSNTAYLQLSSLTSEDTAVYYCARNYPYALDYWGQGTSVTVSS

SEQ ID NO: 15 - DNA, 340 bp, Mus musculus
gacattgtgatgtcacagtctccatcctccctagctgtgtcagttggagagaaggttactctgagctgcaagtccagtcagagccttttatatagtagcgaccaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattataggtatccattcacgttcggctcggggacacagttggaaataaaac

SEQ ID NO: 16 - protein, 113 aa, Mus musculus
DIVMSQSPSSLAVSVGEKVTLSCKSSQSLLYSSDQKNYLAWYQQKPGQSPKLLIYWASTRESGVPDRFTGSGSGTDFTLTISSVKAEDLAVYYCQQYYRYPFTFGSGTQLEIK

SEQ ID NO: 17 - protein, 8 aa, Mus musculus
GFNIEDHY

SEQ ID NO: 18 - protein, 8 aa, Mus musculus
IDPEDGQT

SEQ ID NO: 19 - protein, 10 aa, Mus musculus
ARNYPYALDY

SEQ ID NO: 20 - protein, 12 aa, Mus musculus
QSLLYSSDQKNY

SEQ ID NO: 21 - protein, 9 aa, Mus musculus
QQYYRYPFT